(19) United States
(12) Patent Application Publication
(10) Pub. No.: US 2024/0424244 A1
(43) Pub. Date: Dec. 26, 2024

(54) VALVE

(71) Applicant: Fisher & Paykel Healthcare Limited, Auckland (NZ)

(72) Inventors: Zak Jake FLINTOFF, Auckland (NZ); Johannes Nicolaas BOTHMA, Otorohanga (NZ)

(21) Appl. No.: 18/692,540

(22) PCT Filed: Sep. 21, 2022
(86) PCT No.: PCT/IB2022/058897

§ 371 Date: Mar. 15, 2024

Related U.S. Application Data
(60) Provisional application No. 63/261,439, filed on Sep. 21, 2021.
(60) Provisional application No. 63/366,660, filed on Jun. 20, 2022.

(51) Int. Cl. A61M 16/20; G05D 16/10

(52) U.S. Cl. CPC A61M 16/201; G05D 16/103

(57) ABSTRACT

A valve for use with a respiratory system arranged to convey a breathable gas to a patient. wherein the valve allows gas from within the respiratory system to exit, comprising: a valve body including an inlet and an outlet. said inlet configured to be in fluid communication with the respiratory system: an actuator disposed within the valve body. in a flow path between the inlet and the outlet. wherein the actuator is biased towards the inlet and movement of the actuator away from the inlet being at least partially dependent on a pressure of the gas at the inlet. said movement adjusting the flow path between the inlet and the outlet. to regulate the pressure of the gas in the respiratory system within a predetermined range.

### CROSS REFERENCE TO RELATED APPLICATIONS

This application claims priority to U.S. Provisional Patent Application No. 63/261,439,filed on 21 Sep. 2021 and U.S. Provisional Patent Application No. 63/366,660, filed on 20 Jun. 2022, the disclosures of which are incorporated herein by reference in entirety.

### TECHNICAL FIELD

The present disclosure relates to various devices, systems, and methods applicable to a respiratory system arranged to deliver a breathable gas to a patient. In at least one aspect, the present disclosure relates to a valve for use with a respiratory system. In at least another aspect, the present disclosure relates to a pressure regulating device for facilitating regulating pressure of gases supplied to a patient.

### BACKGROUND

Positive End Expiratory Pressure (PEEP) and/or Peak Inspiratory Pressure (PIP) can be controllably provided to a patient during respiration, resuscitation or assisted respiration (ventilation) by a respiratory system.

PEEP is a pressure delivered to the patient during the expiratory phase of positive pressure ventilation, resuscitation, or assisted respiration. PIP is a desired highest pressure provided to the patient during the inspiratory phase of positive pressure ventilation, resuscitation, or assisted respiration. The patients may be neonates or infants who require breathing assistance or resuscitation. In applying PEEP or PIP, the patient's upper airway and lungs are held open by the applied pressure, and any fluid trapped in the lungs of the patient is cleared or reduced by the flow of air that is supplied to the patient.

Some existing respiratory systems are susceptible to flow variations that can prevent the target PEEP pressure from being achieved. These flow variations can be caused by unintentional leaks or auto-PEEP (also known as inadvertent PEEP), that are not taken into account during system calibration. Unintentional leaks may occur due to non-conforming seals on patient interfaces, incorrect patient interface sizing and fit, patient coughing, movement, and similar thereof. Unintentional leaks can undesirably lower the pressure of the breathable gas delivered to the patient during respiratory therapy.

Auto-PEEP is a phenomenon that occurs when a patient is being actively ventilated, with pressures being alternated between PIP and PEEP, and the patient is still exhaling from the previous inflation when the next inflation is started. This is likely to occur when the expiratory time (i.e. time between inflations) is short, exhaled volumes are large, or there is a resistance to expiratory gas flow. A potential effect of auto-peep is that the pressure that the patient receives during respiratory therapy is higher at the end of the expiratory phase than the targeted PEEP.

Any reference to or discussion of any document, act or item of knowledge in this specification is included solely for the purpose of providing a context for the present invention. It is not suggested or represented that any of these matters or any combination thereof formed at the priority date part of the common general knowledge, or was known to be relevant to an attempt to solve any problem with which this specification is concerned.

### SUMMARY OF THE INVENTION

In a first aspect, the present disclosure provides a valve for use with a respiratory system arranged to convey a breathable gas to a patient, wherein the valve allows gas from within the respiratory system to exit, comprising:
a valve body including an inlet and an outlet, said inlet configured to be in fluid communication with the respiratory system;
an actuator disposed within the valve body, in a flow path between the inlet and the outlet,
wherein the actuator is movable by the gas, movement of the actuator being at least partially dependent on a pressure of the gas at the inlet, wherein said movement adjusts the flow path between the inlet and the outlet, to regulate the pressure of the gas in the respiratory system within a predetermined range.

In some embodiments, the actuator is biased towards the inlet of the valve.

In a second aspect, the present disclosure provides a valve for use with a respiratory system arranged to convey a breathable gas to a patient, wherein the valve allows gas from within the respiratory system to exit, comprising:
a valve body including an inlet and an outlet, said inlet configured to be in fluid communication with the respiratory system;
an actuator disposed within the valve body, in a flow path between the inlet and the outlet,
wherein the actuator is biased towards the inlet and movement of the actuator away from the inlet being at least partially dependent on a pressure of the gas at the inlet, said movement adjusting the flow path between the inlet and the outlet, to regulate the pressure of the gas in the respiratory system within a predetermined range.

In some embodiments, the actuator is caused to move away from the inlet when the pressure exceeds a selected pressure level.

In some embodiments, the predetermined range is a predetermined PEEP pressure range.

In some embodiments, the selected pressure level is within the predetermined PEEP pressure range.

In a third aspect, the present disclosure provides a valve for use with a respiratory system arranged to convey a breathable gas to a patient, wherein the valve allows gas from within the respiratory system to exit, comprising:
a valve body including an inlet and an outlet, said inlet configured to be in fluid communication with the respiratory system;
an actuator disposed within the valve body, in a flow path between the inlet and the outlet,
wherein the actuator is biased towards the inlet, the actuator being caused to move away from the inlet when the pressure exceeds a selected pressure level, said movement adjusting the flow path between the inlet and the outlet, to regulate the pressure of the gas in the respiratory system within a predetermined PEEP pressure range, wherein the selected pressure level is within the predetermined PEEP pressure range.

In some embodiments, the valve comprises a biasing member, operatively coupled to the actuator.

In some embodiments, the actuator is biased towards the inlet by the biasing member.

In some embodiments, the biasing member applies a variable resistance force onto the actuator during movement thereof.

In some embodiments, the variable resistance force applied by the biasing member counters a force exerted onto the actuator caused by the gas pressure at the inlet.

In some embodiments, the flow path between the inlet and the outlet of the valve is closed off, when the pressure at the inlet is below the selected pressure level.

In some embodiments, when the pressure at the inlet is above the selected pressure level, it overcomes the variable resistance force applied onto the actuator by the biasing member, and opens up the flow path between the inlet and the outlet of the valve.

In some embodiments, the biasing member biases the actuator against a portion of the valve body when the flow path is closed off.

In some embodiments, the portion of the valve body forms a valve seat for the actuator.

In some embodiments, the flow path between the inlet and the outlet of the valve is opened up when the actuator is displaced from the valve seat.

In some embodiments, the flow path is at least in part determined by a relative displacement of the actuator from the valve seat.

In some embodiments, the flow path increases its size as the actuator is displaced further away from the valve seat.

In some embodiments, the flow path decreases its size as the actuator moves closer to the valve seat.

In some embodiments, the valve includes a supporting member for the actuator, for guiding and stabilising the movement of the actuator.

In some embodiments, the supporting member is an elongate shaft, along which the actuator is arranged to slide during its movement.

In some embodiments, the actuator includes an orifice, for receiving the shaft therein.

In some embodiments, the orifice and a transverse cross section of the shaft are configured to have a substantially similar shape and size.

In some embodiments, the orifice has a diameter of approximately 1-5 mm.

In some embodiments, the orifice is formed at or near a centre of the actuator.

In some embodiments, the actuator comprises a substantially planar lower surface.

In some embodiments, the actuator comprises a circular disk, wherein the circular disk comprises a substantially planar lower surface.

In some embodiments, the dimension, or an area of the circular disk closely matches the dimension of the valve inlet.

In some embodiments, the actuator comprises a portion for engaging the biasing member.

In some embodiments, the portion comprises a raised portion which extends above an upper surface of the actuator.

In some embodiments, the raised portion extends at least partially into an internal space of the biasing member.

In some embodiments, the valve body defines a chamber, wherein the inlet is formed at or near one end of the chamber, and the outlet is formed at or near another end of the chamber.

In some embodiments, the valve body comprises a housing, and an outlet member.

In some embodiments, the outlet member is arranged to couple to an end of the housing.

In some embodiments, the outlet member is removably coupled to an end of the housing.

In some embodiments, the outlet member includes threaded portions, configured to couple to complimentary threaded portions formed in housing.

In some embodiments, the housing comprises a hollow cylindrical shape, and the valve seat is formed on an interior wall of the housing.

In some embodiments, the outlet member is integrally formed, or permanently connected to the housing.

In some embodiments, the outlet member comprises at least one outlet member orifice, to vent the gas received at the inlet of the valve to ambient air.

In some embodiments, the outlet member orifice has a minimum area of approximately 20 mm².

In some embodiments, the outlet member orifice forms the outlet of the valve.

In some embodiments, the outlet of the valve is arranged to be occluded, when delivering PIP to the patient.

In some embodiments, the outlet of the valve is arranged to be occluded by a finger or a digit of an operator during PIP delivery.

In some embodiments, the outlet of the valve is not occluded, when delivering PEEP to the patient.

In some embodiments, the shaft is received by an orifice of the actuator at a first end, and connected to a portion of the valve body at a second end.

In some embodiments, the outlet member includes one or more arms disposed at or near the outlet member orifice, wherein the second end of the shaft is connected to the one or more arms.

In some embodiments, the outlet member includes a plurality of arms which extend radially outwardly from a centre of the outlet member.

In some embodiments, the biasing member is a spring.

In some embodiments, the biasing member is a coil spring.

In some embodiments, the biasing member is a conical coil spring.

In some embodiments, the biasing member is maintained in a compressed state, when the actuator is biased toward the valve seat.

In some embodiments, the biasing member is compressed further, as the actuator is lifted off the valve seat.

In some embodiments, the variable resistance applied onto the actuator by the biasing member is at least partially dependent on: spring constant, compression of the spring, and/or displacement of the actuator from the valve seat.

In some embodiments, the variable resistance applied onto the actuator by the biasing member may be calculated from F_{bias}=k*(x_initialCompression+x_lift), where
• k=spring constant of the spring;
• x_initial compression=initial compressed length of the spring, when the flow path is closed off (i.e. a difference between the original uncompressed length of the spring, and the length of the spring after it has been initially compressed and placed within the valve body);
• x_lift=displacement of the actuator from the valve seat, at pressure P.

In some embodiments, the gas pressure at the inlet applies a lifting force (F_{lift}) onto the actuator, in a direction which is opposite to the variable resistance applied onto the actuator by the biasing member.

In some embodiments, the lifting force may be calculated from F_{lift}=P*A_{actuator}, where
• P=gas pressure at the valve inlet;
• A_{actuator}=area of the actuator exposed to the gas pressure at the valve inlet.

In some embodiments, the movement of the actuator is determined by the relative relationship of F_{bias} and F_{lift}.

In some embodiments, the actuator is displaced from the valve seat, when F_{lift} is greater than F_{bias}.

In some embodiments, the actuator starts moving closer to the valve seat, when F_{lift} is smaller than F_{bias}.

In some embodiments, the spring constant is at least in part determined by one or more of: spring wire diameter, free length of the spring, diameter of spring coil, number of spring coils, solid height of the spring, spring pitch, and similar thereof.

In some embodiments, the spring constant is smaller than 0.05 N/mm.

In some embodiments, the spring constant is between 0.005 to 0.02 N/mm.

In some embodiments, the biasing member is configured to be retained in a compressed state between the actuator and outlet member, when the actuator engages the valve seat.

In some embodiments, the outlet member is used to adjust the compression of the spring, by adjusting a distance of the outlet member with respect to the actuator.

In some embodiments, the compression of the spring is adjusted by moving the outlet member closer or further away from the actuator.

In some embodiments, the compression of the spring is adjusted by twisting the outlet member in a first or a second direction, wherein when twisting the outlet member in the first direction, the outlet member moves closer to the actuator and compresses the spring further, and when twisting the outlet member in the second direction, the outlet member moves further away from the actuator and reduces the compression of the spring.

In some embodiments, the twisting of the outlet member adjusts the predetermined range of pressure which the valve is configured to regulate.

In some embodiments, the pressure at the inlet of the valve is at least in part determined by the pressure of the gas within the respiratory system.

In some embodiments, the pressure at the inlet of the valve is substantially the same as the pressure of the gas within the respiratory system.

In some embodiments, the pressure of the gas within the respiratory system is at least in part determined by flow variations through the valve caused by unintentional leaks and/or patient's breathing, which causes the pressure to be lower or higher than a targeted PEEP pressure to be delivered to the patient.

In some embodiments, the valve is arranged to compensate for the unintentional leaks, by reducing the gas flow path size through the valve.

In some embodiments, the valve is arranged to compensate for patient's breathing, by allowing a variable portion of the gas within the respiratory system to flow through the valve and exit the respiratory system.

In some embodiments, the respiratory system is configured to deliver a flow rate within a range of 1 L/min to 150 L/min, or 20 L/min to 70 L/min, or up to around 50 L/min, or up to around 30 L/min for adult patients, and a flow rate within a range of 5-15 L/min of breathable gas to a neonatal patient when delivering respiratory therapy.

In some embodiments, the predetermined PEEP pressure range is between 5 and 15 cm H₂O.

In some embodiments, the movement of the actuator is able to regulate the pressure of the breathable gas within the respiratory system by a variation of −2 to +2 cm H₂O.

In some embodiments, the movement of the actuator is able to regulate the pressure of the breathable gas within the respiratory system by a variation of −1 to +1 cm H₂O.

In some embodiments, the movement of actuator is able to regulate the pressure of the breathable gas within the respiratory system by a variation of −0.5 to +0.5 cm H₂O.

In some embodiments, the selected pressure level is within a range of 4.5 to 5.5 cm H₂O.

In some embodiments, the selected pressure level is within a range of 4 to 6 cm H₂O.

In some embodiments, the selected pressure level is within a range of 3 to 7 cm H₂O.

In some embodiments, the selected pressure level is within a range of 6 to 10 cm H₂O.

In some embodiments, the valve is configured to be removably attached to a venting orifice of the respiratory system.

In some embodiments, the venting orifice is provided in a T-piece device.

In some embodiments, the venting orifice is provided in an expiratory conduit of a CPAP device.

In some embodiments, the valve comprises an adaptor, allowing it to be detachably coupled to the respiratory system.

In some embodiments, the valve is configured to be detachably coupled to a T-piece device.

In some embodiments, the valve is configured to be detachably coupled to an expiratory conduit of a CPAP device.

In a fourth aspect, the present disclosure provides a device for use with a respiratory system arranged to convey a breathable gas to a patient, comprising:
a housing including an inlet, an outlet, and a vent, said inlet configured to be in fluid communication with the respiratory system to receive a flow of gas therefrom, said outlet configured to be in fluid communication with a patient interface;
a valve according to the first, second, or third aspect of the present disclosure, wherein the valve is configured to be removably attached to, or integrally formed with the vent of the housing.

In some embodiments, the device is a T-piece resuscitator device.

In some embodiments, the device additionally includes an opening, which may include an optional valve, such as a duckbill valve, for insertion of an auxiliary equipment such as a catheter for fluid clearance or surfactant delivery to the patient.

In some embodiments, the device comprises an adaptor, allowing it to be detachably coupled to the respiratory system.

In some embodiments, the device is arranged to be detachably coupled to an expiratory conduit of a CPAP device.

In a fifth aspect, the present disclosure provides a kit of parts for use with a respiratory system, the kit of parts comprising:
a valve according to the first, second or third aspect of the present disclosure; and
a T-piece device, wherein the valve is connectable to a vent of the T-piece device.

In some embodiments, the kit of parts includes a patient interface, connectable to an outlet of the T-piece device.

In some embodiments, the patient interface includes a range of different sizes and/or fit.

In some embodiments, the kit of parts includes a flexible hose, connectable to an inlet of the T-piece device.

In some embodiments, the kit of parts includes one or more conduits, connectable to a respiratory apparatus to receive a flow of breathable gas therefrom.

In some embodiments, the kit of parts includes connectors, for establishing connections between the valve and the T-piece device, and/or between the one or more conduits and the respiratory apparatus, and/or between the T-piece device and the flexible hose.

In some embodiments, the patient interface may be a CPAP interface.

In a sixth aspect, the present disclosure provides a respiratory system for delivering a respiratory therapy to a patient, the respiratory system comprising:
a respiratory apparatus, which supplies a source of breathable gas flow at a targeted pressure and/or flow rate;
a conduit assembly connectable to the respiratory apparatus to receive the breathable gas flow;
a patient interface, arranged to receive the breathable gas and usable to deliver the respiratory therapy to the patient;
a device arranged to form a fluid connection between the conduit assembly and the patient interface; and
a valve according to the first, second, or third aspect of the present disclosure.

In some embodiments, the respiratory system is connectable to a gas source, which can be a wall mounted gas supply.

In some embodiments, the respiratory system may additionally include a humidifier, for humidifying the breathable gas before it is conveyed to the patient.

In some embodiments, the device includes a housing, including:
an inlet arranged to receive the breathable gas from the respiratory apparatus;
an outlet configured to be in fluid communication with an inlet of the patient interface;
a vent arranged to allow gas from within the respiratory system to exit from the respiratory to ambient air.

In some embodiments, the valve is connectable to the vent of the device.

In a seventh aspect, the present disclosure provides a device for facilitating regulating pressure of gases supplied to a patient, the device comprising:
a housing defining a chamber, the housing having an inlet configured for connection with a gas flow source providing a flow of gases to the chamber, an outlet configured to direct gases out from the chamber, and a vent comprising an actuator for controlling venting of gases from the chamber through the vent;
a biasing member which biases the actuator towards a seated position,
wherein the vent and the actuator are mutually adapted so that the actuator has an exposed area which is exposed to the gases in the chamber, wherein the biasing member comprises a spring constant selected relative to the exposed area of the actuator whereby the actuator remains in the seated position until pressure of the gases in the chamber exceeds a selected pressure level.

In some embodiments, the spring constant may be within a range of 0.005 N/mm to 0.02 N/mm.

In some embodiments, the exposed area of the actuator may be between 70 mm² to 320 mm², or within a range of 100 mm² to 250 mm², or within a range of 120 mm² to 200 mm², or within the range of 140 mm² to 180 mm².

In some embodiments, the exposed area may be, for example about 160 mm².

In some embodiments, the device may be configured for supplying gases to a patient at a flow rate of between 5 L/min and 15 L/min.

In some embodiments, the device may be configured for supplying gases to a patient at a flow rate of about 12 L/min.

In some embodiments, where the patient is an adult the pressure regulating device may be configured to accommodate a flow rate within a range of 1 L/min to about 150 L/min, or about 20 L/min to 70 L/min, or up to around 50 L/min, or up to around 30 L/min.

In some embodiments, the selected pressure level may be positive end expiratory pressure (PEEP), within a range of between 5 to 15 cm H₂O, or within a range of 6 to 10 cm H₂O, or 4 cmH₂O to 8 cmH₂O, or 3 cmH₂O to 7 cmH₂O.

In some embodiments, the selected pressure may be peak inspiratory pressure (PIP) within a range of between 20 cmH₂O and 80 cmH₂O, or within a range of between 20 cmH₂O and 40 cmH₂O.

In some embodiments, the vent and the actuator are mutually adapted so that the actuator moves along a venting axis when moving relative to the seated position.

In one embodiment, the pressure regulating device comprises an outlet member associated with the vent, the outlet member comprising a supporting member aligned with the venting axis, the actuator having an orifice formed therein which accommodates the supporting member so that the actuator moves along the supporting member when moving relative to the seated position.

In some embodiments, the supporting member may comprise a shaft configured to extend through the orifice, the orifice extending through the actuator, so that the actuator moves relative to the shaft.

In some embodiments, the orifice may be a blind bore.

In some embodiments, the shaft and the orifice are mutually dimensioned within a tolerance range of 0.15 mm to 0.025 mm so that any flow of gases through a gap formed between the shaft and the orifice is negligible.

In some embodiments, the vent may be configured to position the outlet member downstream from the actuator, the outlet member having an orifice through which gases pass when venting, the orifice being configured relative to the actuator such that the outlet member provides less restriction to the flow of gases venting through the venting outlet than the actuator when moved away from the seated position.

In some embodiments, the orifice is configured for being selectively occluded such as by placement of a digit of a user thereover.

In some embodiments, the actuator and the outlet member may be configured to position the biasing member therebetween.

In some embodiments, the biasing member may be adapted to extend coaxially with venting axis.

In some embodiments, the outlet member is adapted to engage a first end of the biasing member so as to inhibit movement of the biasing member in a direction not aligned with the venting axis.

In some embodiments, the actuator is adapted to engage a second end of the biasing member so as to also inhibit movement of the biasing member in a direction not aligned with the venting axis.

In some embodiments, the actuator may be annular in shape.

In some embodiments, the actuator and the vent are mutually adapted for gases venting from the chamber to egress evenly over a perimeter of the actuator so that the pressure remains evenly spread over the exposed surface when the actuator moves from the seated position.

In some embodiments, the vent and the outlet member are mutually adapted for engagement therebetween.

In one arrangement, the vent comprises a threaded inner surface and the outlet member comprises a threaded outer surface so that the threaded engagement allows the outlet member to locate in the vent to thereby discourage manual adjustment of the outlet member relative to the venting outlet when the device is in use.

In another arrangement, the vent comprises an outlet member configured to interact with the biasing member for adjusting a percentage of compression of the biasing member while the actuator is in the seated position.

In some embodiments, the vent is formed integrally with the outlet member, the vent comprises a threaded outer surface and the outlet member comprises a threaded inner surface so that the threaded engagement allows manual adjustment of the outlet member relative to the vent when the device is in use.

In some embodiments, the engagement between the vent and the outlet member may be through welding, clipping or gluing.

In some embodiments, the biasing member may be a conical coil spring, a coil spring, or other forms of spring.

In some embodiments, the housing may additionally comprise an aperture configured to allow insertion of equipment therethrough, such as surfactant delivery means or a suction tube.

In some embodiments, where the housing includes an aperture, a sealing means is provided which is configured to prevent gas flow through said opening and allow the equipment to be inserted therethrough while providing gases to a patient.

In one embodiment, the sealing means may be a duck bill valve, however other forms of valve may also be suitable.

In some embodiments, the opening may be located at any suitable location on the housing, and in one arrangement the housing is substantially aligned with the outlet on an opposed side of the chamber.

In some embodiments, the vent may comprise a valve seat which extends radially and is engaged by a radial perimeter surface of the actuator when in the seated position, so that an engaged area between the valve seat and the radial perimeter surface is within a radial dimension within a range of 0.2 mm to 2.0 mm.

In some embodiments, the actuator may be relatively rigid so that it disengages uniformly from the valve seat when moved from the seated position.

In some embodiments, the actuator may be formed from any suitable material, including a thermoplastic elastomer such as polyurethane, silicon, or rubber.

In an eighth aspect, the present disclosure provides a respiratory system for facilitating delivering gases to a patient, the respiratory system comprising, at least one device according to the seventh aspect, a gas flow source configured to supply a flow of gases, and a patient interface configured to engage a patient.

In some embodiments, the gas flow source may provide a flow of gases at a flow rate in a range of about 1 L/min to about 150 L/min, or about 20 L/min to about 70 L/min, or up to around 50 L/min, or up to around 30 L/min.

In some embodiments, the gas flow source may provide a flow of gases at a flow rate in a range of about 5 to 15 L/min.

In some embodiments, the respiratory system may include a controller for controlling the flow of gases from the gas flow source supplied to the device.

In some embodiments, the controller may receive inputs indicative of pressure in the patient's airway and adjust control of the gas flow source to set a peak inspiratory pressure (PIP).

In some embodiments, the controller may receive inputs indicative of pressure in the patient's airway and adjust control of the gas flow source to set a continuous positive airway pressure (CPAP).

In some embodiments, the patient interface may be any suitable interface such as a mask, endotracheal tube, laryngeal mask, or nasal cannula.

In some embodiments, the respiratory system may also comprise a humidifier configured to condition the gas to a pre-determined temperature and/or humidity before delivery to the patient.

In some embodiments, the respiratory system may comprise a gas delivery conduit providing a flow of gas from the gas flow source to at least the patient interface.

In some embodiments, the respiratory system may comprise a plurality of devices.

In a nineth aspect, the present disclosure provides a pressure regulating device for facilitating regulating pressure of gases supplied to a patient, the device comprising:
a housing defining a chamber, the housing comprising an inlet couplable with a flow source providing a flow of gases to the chamber, an outlet couplable with a patient interface for supplying gases to the patient from the chamber, and a vent comprising an actuator for controlling venting of gases from the chamber through the vent;
a biasing member which biases the actuator towards a seated position whereby the actuator remains in the seated position until pressure of the gases in the chamber exceeds a selected pressure level, and
an outlet member associated with the vent, wherein the outlet member comprises a supporting member aligned with a venting axis, the actuator having an orifice formed therein which accommodates the supporting member so that the actuator moves along the supporting member when moving relative to the seated position.

In some embodiments, the supporting member may comprise a shaft which locates within the orifice, the orifice extending through the actuator, so that the actuator moves relative to the shaft.

Further features and advantages of the present disclosure will become apparent from the following detailed description.

### BRIEF DESCRIPTION OF THE DRAWINGS

Various preferred embodiments of the present disclosure will now be described, by way of examples only, with reference to the accompanying figures, in which:

FIG. 1 illustrates an example of a respiratory system;

FIG. 2 shows another example of a respiratory system;

FIG. 3 shows an example of a T-piece device in use, with its PEEP orifice occluded;

FIG. 4 shows another view of the T-piece device in use, with its PEEP orifice unoccluded;

FIG. 5 shows another example of a T-piece device in use, with its PEEP orifice occluded;

FIG. 6 shows another view of the T-piece device in use, with its PEEP orifice unoccluded;

FIG. 7 illustrates directions of gas flows in a T-piece device;

FIG. 8 shows a cross sectional diagram of an existing PEEP valve;

FIG. 9 shows a cross sectional diagram of an existing PIP valve;

FIG. 10 shows a cross sectional diagram of a valve according to an embodiment of the present disclosure;

FIG. 11 shows a cross sectional diagram of the valve of FIG. 10, with the actuator of the valve being lifted from its valve seat;

FIG. 12 shows an exploded perspective view of the valve of FIGS. 10 and 11, configured to be used with a T-piece device;

FIG. 13 shows a side cross sectional diagram of the valve and the T-piece of FIGS. 10 and 11, with the valve coupled to a PEEP orifice of the T-piece device;

FIG. 14 shows a cross sectional schematic of a valve and parameters of its components;

FIG. 15 shows an exploded perspective view of another embodiment of a valve according to the present disclosure;

FIG. 16 shows the valve of FIG. 15 connected to a T-piece device;

FIG. 17 shows an exploded side perspective view of a further embodiment of a valve according to the present disclosure;

FIG. 18 shows a perspective view of the valve of FIG. 17, configured for coupling with a T-piece;

FIG. 19 shows a bottom perspective view of a body of the valve of FIGS. 17 and 18;

FIG. 20 shows a cross sectional schematic of the valve of FIGS. 17 and 18;

FIG. 21 shows a side view of the valve of FIGS. 17 and 18, after it is coupled to a T-piece;

FIG. 22 shows an exploded side perspective view of a further embodiment of a valve according to the present disclosure;

FIG. 23 shows a perspective view of the valve of FIG. 22, configured for coupling with a T-piece;

FIG. 24 shows a side view of the valve of FIGS. 22 and 23, after it is coupled to a T-piece device;

FIGS. 25, 26, 27 illustrate an example of using a valve of the present disclosure with a respiratory system including a CPAP interface;

FIGS. 28, 29, 30 illustrate a further example of using a valve of the present disclosure with a respiratory system including a CPAP interface;

FIG. 31 shows a comparison of a pressure curve with auto-PEEP and a pressure curve without auto-PEEP;

FIG. 32 shows a comparison of pressure curves generated by a theoretical “perfect system” and by a respiratory system including a valve of the present disclosure;

FIG. 33 demonstrates the pressure regulating effect of the present valve compensating for unintentional leaks of the respiratory system.

### DETAILED DESCRIPTION OF EMBODIMENTS

The present disclosure relates to various devices, systems, and methods applicable to a respiratory system arranged to deliver a breathable gas to a patient. In at least one aspect, the present disclosure relates to a valve for use with a respiratory therapy system. In at least one aspect, the present disclosure provides a pressure regulating device for facilitating regulating pressures of gases supplied to a patient.

The respiratory therapy mentioned throughout this disclosure can be resuscitation therapy, such as infant or neonate resuscitation therapy, positive airway pressure therapy (PAP), continuous positive airway pressure therapy (CPAP), bi-level positive airway pressure therapy, non-invasive ventilation, or another form of respiratory therapy. In some configurations, the system may provide bi-level positive airway pressure therapy to achieve infant resuscitation.

‘Pressure therapy’ as used in this disclosure may refer to delivery of a breathable gas to a patient at a pressure of at least greater than or equal to about 1 cmH₂O. Pressure therapy may be delivered to mimic natural breathing cycles of a patient, and/or delivered in accordance with the patient's breathing cycles to assist with the patient's breathing.

In some configurations, the breathable gas delivered to the patient is, or comprises, oxygen. In some configurations, the breathable gas comprises a blend of oxygen or oxygen enriched gas, and ambient air. In some configurations, the percentage of oxygen in the gases delivered may be between about 20% and about 100%, or between about 30% and about 100%, or between about 40% and about 100%, or between about 50% and about 100%, or between about 60% and about 100%, or between about 70% and about 100%, or between about 80% and about 100%, or between about 90% and about 100%, or about 100%, or 100%. In at least one configuration, the gases delivered may be of atmospheric composition. In at least one configuration, the gases delivered may be ambient air.

In relation to infant resuscitation, when in utero, the lungs of a foetus are filled with fluid, and oxygen comes from the blood vessels of the placenta. At birth, the transition to continuous postnatal respiration occurs, assisted by compression of the lungs by the birth canal. Also assisting the infant to breathe is the presence of surfactant that lines the alveoli to lower surface tension, preventing the alveolar walls from sticking to each other.

Any newborn may require respiratory assistance to either begin or improve breathing at birth. However, several factors may predict the need for resuscitation or respiratory assistance during the transition to continuous postnatal respiration. For example, birth at less than 35 weeks' gestational age, evidence of significant foetal compromise, maternal infection, or congenital abnormality and emergency caesarean deliveries are associated with an increased need for respiratory assistance at birth.

### 1. Overview

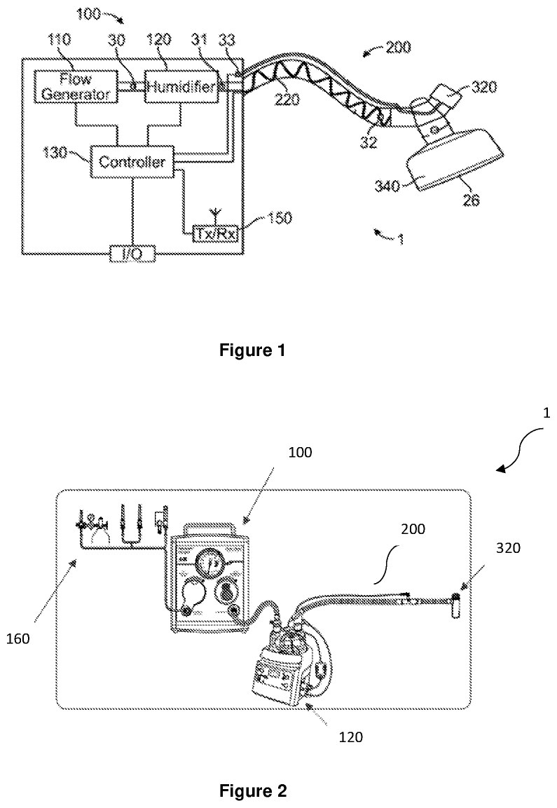

An example of a respiratory system 1 is shown in FIG. 1. Another example of a respiratory system 1 is shown in FIG. 2. The respiratory system 1 is configured to provide respiratory therapy to a patient, by delivering a breathable gas to an airway of the patient.

In general terms, the respiratory system 1 comprises a respiratory apparatus 100, a conduit assembly 200 arranged to convey a breathable gas from the respiratory apparatus 100 to a patient, and a patient interface 340 arranged to be in communication with an airway of a patient. Some respiratory systems may additionally include a device 320 configured to fluidly connect to the patient interface 340 when delivering respiratory therapy. In at least some embodiments, the device 320 includes suitable connectors allowing it to fluidly couple to an inlet of the patient interface 340 at one end, and fluidly couple to a connector of the conduit assembly 200 at another end.

With reference to FIG. 1, the respiratory therapy apparatus 100 may include a flow generator 110, an optional humidifier 120 for humidifying the gases generated by the flow generator 110, and an associated controller 130 which manages operation of the flow generator 110 and/or the humidifier 120, when present. In at least one embodiment, the flow generator 110 may be in the form of a blower.

The respiratory therapy apparatus 100 may also comprise a transmitter 150, receiver 150, and/or transceiver 150 to enable the controller 130 to receive transmitted signals from the sensors 30, 31, 32, 33 and/or to control the various components of the respiratory system 1, such as the flow generator 110, humidifier 120, humidifier heating element 220, or accessories or peripherals associated with the respiratory therapy apparatus 100.

FIG. 2 shows another example of a respiratory system 1, including a respiratory therapy apparatus 100, which may be a positive pressure ventilation device such as a resuscitator. An example of a resuscitator is the Fisher and Paykel Healthcare Neopuff™ Infant Resuscitator. The respiratory therapy apparatus 100 receives a flow of breathable gas from a gas supply source 160 via a gas inlet. The respiratory therapy apparatus 100 may be connected to an optional humidifier 120 via a gas outlet of the apparatus 100. The humidified breathable gas is then supplied to the patient from an outlet of the humidifier 120 via a conduit assembly 200 and a device 320, which is connectable to a patient interface (not shown). The gas supply source 160 usually supplies the flow of breathable gas at a substantially constant flow rate to the respiratory apparatus 100. The respiratory apparatus 100 receives the flow of breathable gas and is usually configured in an initial calibration phase to set the level of pressures to be delivered to the patient. The level of pressures may be PIP and/or PEEP.

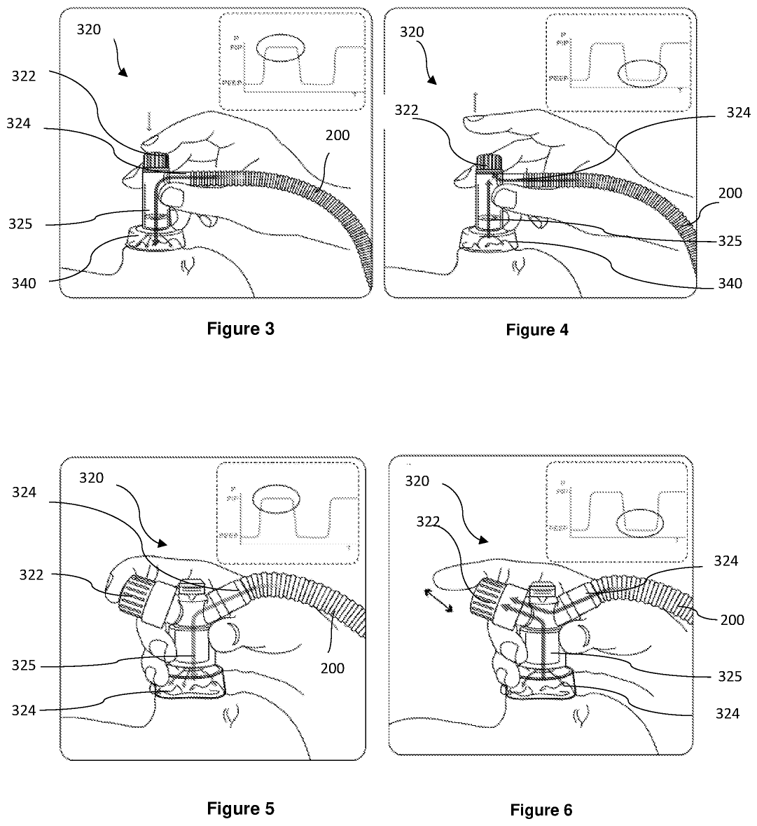

As mentioned above, the device 320 is provided for use with the respiratory system 1, and when in use, it fluidly connects the conduit assembly 200 to the patient interface 340. In some existing respiratory systems, the device 320 is used by an operator of the system to manually adjust the pressure of gas delivered to the patient, as illustrated in FIGS. 3 to 6.

With reference to FIGS. 3 to 6, each device 320 includes an inlet 324 arranged to receive the breathable gas from the respiratory apparatus 100. An outlet 325 of the device 320 is arranged to be fluidly connected to the patient interface 340 when delivering respiratory therapy. Each device 320 also includes a PEEP vent 322 arranged to be occluded or unoccluded with a finger or a digit of an operator when delivering respiratory therapy to the patient. When the PEEP vent 322 is occluded by the operator, the breathable gas received from the respiratory apparatus 100 is delivered to the patient via the patient interface 340, and the respiratory system 1 delivers the breathable gas at a second pressure to a patient. When the occlusion is removed from the PEEP vent 322, the PEEP vent 322 allows gas from within the respiratory 1 to exit from an internal cavity of the device 320 to ambient air, and the respiratory 1 delivers the breathable gas at a first pressure to the patient. In this way, resuscitation of a patient can be attempted by varying between the first and second pressures at a selected breathing rate.

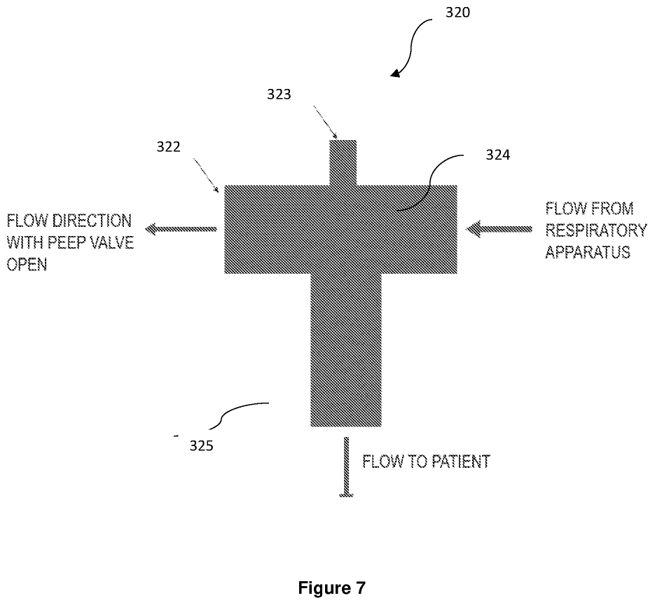

FIG. 7 illustrates flow directions of the breathable gas as it enters the device 320 via its inlet 324, and exits the device 320 from the PEEP vent 322 (if it is not occluded), and/or from the outlet 325, which is connected to the patient interface 340 when in use. An optional valve 323, such as a duckbill valve, may also be included in the devices shown in FIGS. 5 and 6, which can be used for insertion of an auxiliary equipment such as a catheter for fluid clearance or surfactant delivery, or a gas detection device, such as a CO₂ detector.

The configuration of the devices 320 in FIGS. 3 to 6 allows for one handed manual operation during resuscitation therapy. Overtime, the varying pressures of breathable gas conveyed to the patient may be represented by a roughly square waveform, indicated in top right hand corners of FIGS. 3 to 6.

In some embodiments, the first pressure level is delivered at or near the patient terminal end 26 at a first time or during a first time window. The first pressure level may be delivered at or near the patient terminal end 26 once interface fit is confirmed.

In one embodiment, the first pressure level is equal to desired PEEP. The first pressure may be 1, 2, 3, 4, 5, 6, 7, 8, 9, 10, 11, 12, 13, 14 or 15 cm H₂O, and a useful value may be selected between any of these ranges (for example, about 1 to about 15, about 1 to about 14,about 1 to about 13, about 1 to about 12, about 1 to about 11, about 1 to about 10, about 1 to about 9, about 1 to about 8, about 1 to about 7, about 1 to about 6, about 1 to about 5, about 2to about 8, about 2 to about 6, about 2 to about 5, about 3 to about 8, about 3 to about 5, about 4 to about 8, about 4 to about 7, about 4 to about 5, about 5 to about 8 or about 6 to about 8 cm H₂O). The first pressure may be about 5 cm H₂O, but can be set depending on, for example, patient requirements and/or clinician preference.

Similarly, a second pressure level can be delivered at or near the patient terminal end 26 at a second time or during a second time window. The second pressure level may be delivered at or near the patient terminal end 26 once interface fit is confirmed and/or once intended second pressure level has been confirmed in the resuscitator 100, for example, by sealing the outlet of the device 320 with a protective cover (e.g. during the initial calibration phase). The respiratory system 1 continuously provides the breathable gas to the patient at the first and the second pressure levels in order to mimic patient's breathing cycles. Typically, for infant or paediatric patient populations, 30-60 breathing cycles/minute are provided to the patient during respiratory therapy. In some applications, a patient's breathing cycles are manually determined by a clinician. It should be appreciated that the number of breathing cycles/minute required is largely dependent on the type of therapy to be provided to the patient, patient's condition (age, breathing condition), and may vary from patient to patient, or based on different hospitals' protocol.

In at least one embodiment the second pressure level is equal to desired PIP. The second pressure may be 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 cm H₂O, and a useful value may be selected between any of these ranges (for example about 15 to about 60, about 20 to about 25, about 21 to about 30, about 21 to about 27, about 21 to about 25, about 22 to about 30, about 22 to about 29, about 22 to about 25, about 23 to about 30, about 23 to about 28, about 23 to about 26, about 24 to about 30, about 24 to about 29, about 24 to about 28, about 24 to about 26 or about 25 to about 30 cm H₂O). A higher PIP may be needed for first few breathing cycles (for clearing liquid from airways and beginning lung aeration) and/or if the patient does not respond positively to initially given respiratory therapy. In addition, the level of pressure required for resuscitation usually varies from patient to patient, depending on factors such as maturity of lungs, presence of lung disease, disorder, and similar thereof. The pressure ranges mentioned above are for guide only and in practice pressures can be individually adjusted depending on patient's response.

In at least one configuration, the patient interface 340 can be in the form of a sealed patient interface. In at least one configuration, the patient interface 340 can be in the form of a respiratory mask, or endotracheal tube, or laryngeal mask. The patient interface 340 can be configurated to deliver a breathing gas to the patient's airway via a seal or cushion, at the patient terminal end 26. The patient interface 340 is intended to form an airtight seal in or around the patient's nose and/or mouth. The patient interface 340 can be an oronasal, nasal, direct nasal, and/or oral patient interface, which creates a substantially airtight seal between the patient terminal end 26 and the nose and/or mouth of the patient. In at least one embodiment, the seal or cushion can be held in place on the patient's face by headgear. In at least one embodiment, the patient interface 340 can be held on the patient's face by an operator who may be a healthcare professional. Such sealed patient interfaces can be used to deliver pressure therapy to the patient. Alternative patient interfaces, for example those comprising nasal prongs can also be used. In some examples, the nasal prongs may be sealing or non-sealing. In at least one embodiment, the patient interface is a CPAP interface, such as the ones described in WO2021176338A1, the content of which is incorporated herein in its entirety by reference.

A neonatal interface may be any interface, such as described above, that is configured for use with an infant or neonate. The neonatal interface may be configured to at least partially, and preferably substantially seal around the nose and mouth of the patient.

To provide respiratory therapy to a patient, it is required to regulate the pressure delivered to the patient within a certain range, to ensure effectiveness of the respiratory therapy and also prevent injury to patients. This is particularly relevant to infants and neonates due to the fragility of their lungs and airway. In the past, a number of different valves have been used to achieve such pressure regulation. For example, for the respiratory system 1 illustrated in FIG. 2, the PEEP vent of the device 320 may include a pressure regulating valve (not shown) which allows the gas from within the respiratory system 1 to vent externally and reduce any excess pressure within the respiratory system 1. This pressure regulating valve is usually referred to as a PEEP valve. A pressure regulating valve may also be provided in the respiratory apparatus 100 of FIG. 2, to set the pressure of the breathable gas delivered to the patient at PIP. This is usually referred to as a PIP valve. In addition, a maximum pressure relief valve may also be provided in the respiratory apparatus 100, to set the maximum pressure that may be delivered to the patient. In infant or neonate respiratory therapy systems, the maximum pressure relief may be set to about 40 cmH₂O.

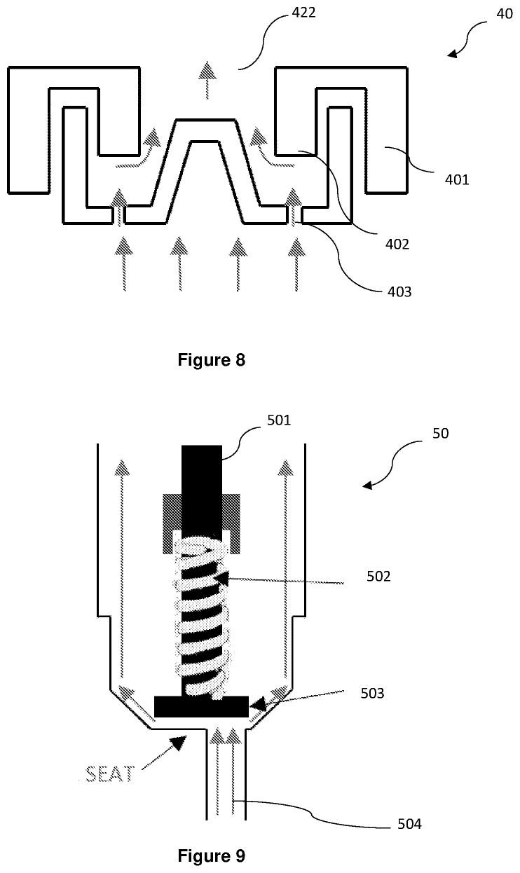

FIG. 8 shows a cross sectional schematic diagram of a known PEEP valve 40. In use, the PEEP valve 40 allows gas to flow through the valve 40 and exit the respiratory system in a direction indicated by the arrows. The PEEP valve 40 is used to provide a manual pressure adjustment, as previously described with reference to FIGS. 3 to 6. That is, when the PEEP vent 422 is occluded, the PEEP valve 40 closes and prevents air from flowing through the valve 40. When the occlusion is removed from the PEEP vent 422, the valve 40 opens which allows gas to flow through the valve in the direction indicated by arrows. Such PEEP valve 40 provides a fixed flow restriction to the gas that is required to flow through the valve 40. In some existing PEEP valves 40, the flow restriction created by the valve is adjustable, by twisting an outlet member 401 of the valve, which adjusts a vertical distance between an inner edge 402 of the outlet member and a gas inlet 403 of the valve 40. A spring may be included to prevent accidental or inadvertent change of PEEP level during operation. Whist this device may be termed a ‘valve’, its operation is as a manually operated fixed flow restriction device and would not be closed at any time during operation, except when its outlet 422 is manually occluded by an operator to switch between PEEP and PIP.

FIG. 9 shows a cross sectional diagram of a known PIP valve 50. The PIP valve 50 includes a plunger 501 which is biased toward a gas inlet 504 of the valve 50, by a spring 502. The lower end of the plunger 501 includes a flat region 503. During the delivery of bi-level resuscitation therapy, the plunger 501 remains seated on the valve seat when PEEP is being delivered, and is lifted off the seat to a fixed distance, when PIP is being delivered. The PIP valve 50 does not provide any pressure regulation effect during PEEP delivery, due to the plunger 502 being seated on the valve seat. Further, the PIP valve 50 is usually located within the resuscitator device 100 and controlled by a controller of the resuscitator device 100, upstream from where the gas is delivered to the patient, so it is not usually subject to flow variations which typically occur near the patient's end (e.g. leak, or an increase in flow caused by patient's breathing).

The present disclosure aims to provide a valve, configured to respond to, and compensate for flow variations experienced by the respiratory system, as will be described further below.

### 2. Valve

According to one aspect of the present disclosure, a valve is provided for use with the respiratory system 1 described above. The valve assists with regulating a pressure of the gases supplied to a patient. In at least some embodiments, the valve assists with regulating the PEEP pressure provided to the patient, such that the gas pressure delivered to the patient remains in a predetermined pressure range, being a PEEP pressure range. In at least some embodiments, the valve may be configured to assist with regulating the PIP pressure provided to the patient, such that the gas pressure delivered to the patient remains in a predetermined pressure range, being a PIP pressure range. According to the present disclosure, the valve achieves its pressure regulation effect by acting as a variable flow restriction of the respiratory system 1, due to a biasing member of the valve being reactive to flow variations, and pressure variances, which are unaccounted for in the initial calibration phase of the respiratory therapy apparatus, but which are often experienced by the respiratory system 1 nearly the patient's end. The valve provides a continuous mechanical adjustment of the flow restriction in accordance with instantaneous gas pressures at an inlet of the valve, without any manual or controller intervention. The flow restriction created by the valve is at least in part determined by a position of an actuator with respect to a valve seat, as will be described further below.

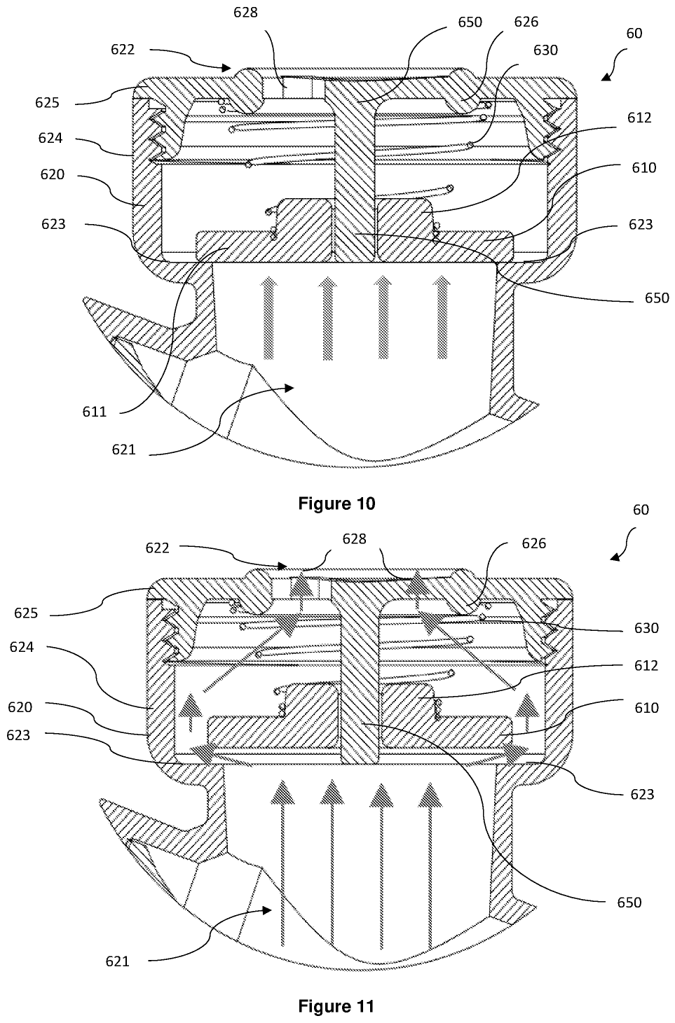

FIGS. 10, 11, 12, 13 show various different views of one embodiment of a valve 60 according to the present disclosure.

With reference to FIG. 10, the valve 60 includes a valve body 620, defining an inlet 621, and an outlet 622, via which a gas flow may enter and exit the valve 60 when the valve is open. In a simplest form, the valve body 620 may include a chamber, and one end of the chamber includes the inlet 621, whereas another end of the chamber includes the outlet 622. In the illustrated embodiment, the valve body 620 comprises at least a housing 624, and an outlet member 625 which is configured to be coupled to the housing 624 via a suitable coupling, such as a screw thread or press fit.

An actuator 610 is accommodated within the valve body 620, in a space created by the housing 624 and the outlet member 625. In other embodiments, the housing 624 and the outlet member 625 may be permanently connected or integrally formed together, without a coupling in between. In the embodiment shown, the coupling between the housing 624 and the outlet member 625 includes threaded portions formed on an inner wall of the housing 624, and corresponding threaded portions formed on an external wall of the outlet member 625 which extends into the housing 624. It will be appreciated that the coupling between the housing 624 and the outlet member 625 may be provided in alternative forms and not necessarily restricted to a thread coupling as illustrated. In at least some embodiments, the coupling allows the housing 624 and the outlet member 625 to be detachably coupled to each other. The relative position of the housing 624 and the outlet member 625 may also be adjusted, via the coupling. For example, when a thread coupling is used, the outlet member 625 may be twisted with respect to the housing 624, to gradually and linearly increase or decrease a height of the internal space of the valve body 620.

In one form, the housing 624 includes a substantially hollow body, which may be formed in a cylindrical shape. At or toward a lower end of the housing 624, the diameter of the cylindrical body reduces, allowing an inner wall of the housing 624 to form a valve seat 623 for the actuator 610, and determine a seated position of the actuator 610.

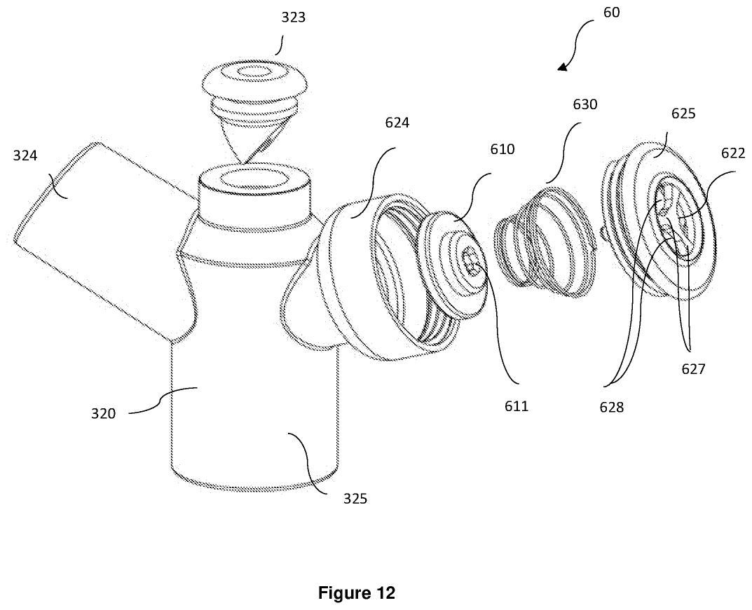

The outlet member 625 may engage the housing 624 at an upper end thereof. As shown in FIG. 12, the outlet member 625 may be formed in a circular shape, including at least one orifice 628 at or near a centre of the outlet member 625. In some embodiments, a plurality of orifices 628 may be formed in the outlet member 625. The orifice 628 forms the outlet 622 of the valve 60.

In some embodiments, a shaft 650 is also accommodated within the valve body 620, and is aligned with a movement direction of the actuator 610. This movement direction may be considered as venting axis of the valve 60. As illustrated, the shaft 650 may have an elongate shape, with its lower end being received in an orifice of the actuator 610. The orifice may be formed at or near a centre of the actuator 610.

In at least one embodiment, the shaft 650 may be formed as an elongate rod having a circular cross section in a transverse direction. The orifice of the actuator 610 may be configured to have a different shape from the cross sectional shape of the shaft 650. For example, a square orifice may be formed in the actuator 610 which the shaft 650 extends into, such that a small gap is present between the shaft 650 and the actuator 610. The orifice may be provided in other shapes, different to the cross sectional shape of the shaft 650 in profile and/or dimension. In some embodiments, the orifice of the actuator 610 may have the same shape as the cross-sectional shape of the shaft 650 but may be larger in size such that a gap is present between the shaft 650 and the actuator 610 when the shaft 650 extends into the actuator 610. Providing a small gap between the shaft 650 and the actuator 610 assists with reducing the friction as the actuator 610 moves along the shaft 650, as well as enabling a controlled leak via the gap.

In some embodiments, the shaft 650 and the orifice of the actuator 610 are configured to reduce gas leaking through the gap formed between the shaft 650 and the actuator 610. In these embodiments, the gap may have a width within a range of 0.025 to 0.15 mm so that any flow of gases through the gap is negligible.

An upper end of the shaft 650 may be supported by the outlet member 625. For example, the outlet member 625 may include one or more arms 627 which are located within outlet 622 and extend radially outwardly from a centre of the outlet member 625, to connect to an inner rim of the outlet 622. Spaces between adjacent arms 627 then form the one or more orifices 628. A receptacle may be formed beneath where the one or more arms are joined, in order to receive the upper end of the shaft 650. In one form, the receptacle may be formed in a square shape, or any other shape. In this way, the shaft 650 is secured in a vertical orientation, to help stabilise movement of the actuator 610 along its venting axis. In at least one embodiment, the upper end of the shaft 650 may be directly connected to the one or more arms 627, or integrally formed with the one or more arms 627.

The shaft 650 is arranged to guide and stabilise the movement of the actuator 610 during its pressure regulation. Without the shaft 650, the actuator 610 may flutter on the valve seat 623, when influenced by factors such as stiffness of a biasing member 630, stability of the actuator 610, and rapid pressure changes within the respiratory system 1. When the actuator 610 flutters, it may create noise and internal pressure oscillations. The shaft 650 assists with stabilising the actuator 610, by allowing it to slide along the axis of the shaft 650 in the vertical direction, while minimising any lateral movement.

The actuator 610 may be configured to have a substantially planar lower surface. In one form, the actuator may include a circular disk 611, which includes a substantially planar lower surface. A peripheral region of the circular disk 611 is configured to engage or rest on the valve seat 623, as indicated in FIG. 10. A substantial portion of the planar lower surface of the circular disk 611 is configured to be exposed to the gas at the inlet 621 of the valve 60, allowing the gas pressure to apply an evenly distributed lifting force (F_{lift}) to the exposed area of the circular disk 611, in an upward direction. In some embodiments, the actuator 610 additionally includes a raised portion 612 with respective to an upper surface of the actuator 610. The raised portion 612 engages a lower end of the biasing member 630, for example, by extending into an internal volume of the biasing member 630. An upper end of the biasing member 630 engages a lower surface of the outlet member 625. In some embodiments, a similar raised portion 626 may also be provided to the lower surface of the outlet member 625, to assist with maintaining a position of the biasing member 630 within the valve body 620. In alternative forms, the actuator 610 may be configured differently from the illustrated embodiment, and not necessarily having a planar lower surface.

In FIG. 10, the actuator 610 is biased toward the inlet 621 of the valve 60 by the biasing member 630. In at least some embodiments, the biasing member 630 may be a spring, such as a coil spring including a plurality of coils. In at least one embodiment, the biasing member is a conical coil spring. That is, a coil at one end of the spring is configured to have a different coil size compared to a coil at another end of the spring. The sizes of the coils may gradually increase from one end of the spring to the other end. In some embodiments, the size of the coils of the conical spring may gradually increase from an end which engages the actuator 610 to an end which engages the outlet member 625. A conical spring may be preferred in some embodiments, so that the spring does not significantly influence the resistance to flow and provides a degree of lateral stability for the valve 60.

The biasing member 630 is held in a compressed state in the valve body 620. That is, the height of biasing member 630 in FIG. 10 is smaller than an uncompressed natural length of the biasing member 630. As the biasing member 630 is already compressed, it applies a resistance force (F_{bias}) onto the actuator 610, which pushes the actuator 610 toward the inlet 621 of the valve 60, until it engages the valve seat 623. When in this seated position, the actuator 610 closes off a gas flow path between the inlet 621 and the outlet 622 of the valve body 620, minimising any gas flow through the valve 60. The resistance force caused by the biasing member may be calculated from F_{bias}=k*(x_initialCompression+x_lift), where
• k=spring constant of the spring;
• x_initial compression=initial compressed length of the spring, when the flow path is closed off (i.e. a difference between the original uncompressed length of the spring, and the length of the spring after it has been initially compressed and placed within the valve body 620);
• x_lift=displacement of the actuator 610 from the valve seat, at pressure P.

It will be appreciated that the resistance force caused by the biasing member is a variable resistance force, due to x_lift (which may change with changes in pressure level P). When the actuator 610 is biased against the valve seat 623, x_lift equals 0, as there is no relative displacement of the actuator 610 from the valve seat 623 yet. The initial variable resistance force applied by the biasing member 630 may be simplified to F_{bias}=k*x_initialCompression. As the actuator 610 is lifted off the valve seat 623, and starts sliding along the shaft 650 with the valve body, x_lift equals the displaced distance of the actuator 610 with respect to the valve seat 623, which is variable. That is, the variable resistance force varies in accordance with movement of the actuator 610, which changes compression of the biasing member 630.

In addition to the variable resistance force (F_{bias}) applied by the biasing member 630, the actuator 610 is also subject to an opposing, upward lifting force (F_{lift}), due to the area of the actuator 610 which is exposed to the gas at the inlet of the valve 60. The lifting force is dependent on the pressure level, P, at the inlet 621, and the area of the actuator exposed to such pressure (F_{lift}=P*A). Accordingly, the position and movement of the actuator 610 with respect to the valve seat 623 is determined by the relative strengths of the two forces F_{bias} and F_{lift}.

Using the equations mentioned above, a minimum pressure that is required to lift the actuator 610 off the valve seat 623 can be determined, which is a pressure greater than k*x_initialCompression/A. This minimum pressure level determines the selected pressure level at which the valve 60 is open, as well as the predetermined pressure range which the valve 60 is configured to regulate. It will be appreciated that the pressure level at the inlet 621 may fluctuate during delivery of breathable gas to the patient, when the flow rate of gases through the valve 60 changes e.g. an increase in the flow rate of gases through the valve 60 will increase the pressure level at the inlet 621, whereas a decrease in the flow rate of gases through the valve 60 may decrease the pressure level at the inlet 621.

If the gas pressure with the respiratory system 1 is lower than the selected pressure level, F_{lift} is not sufficient to lift the actuator 610 off the valve seat 623. Accordingly, the net effect of two forces maintains the actuator 610 in its seated position on the valve seat 623 (F_{bias}≥F_{lift}). As gas pressure at the inlet 621 starts to increase, it translates into an increased F_{lift}. As the gas pressure meets or exceeds the selected pressure level, which causes F_{lift} to be greater than F_{bias}, the lifting force will overcome the variable resistance force applied by the biasing member 630 (F_{lift}>F_{bias}), and lifts the actuator 610 above valve seat 623, as indicated in FIG. 11.

As the actuator 610 is lifted off the valve seat 623, a gap is formed between the peripheral region of the actuator 610, and the valve seat 623. The gas flow may then start to enter the valve body 620 through this gap, and flows out of the valve via the outlet 622. In other words, the gas flow path between the inlet 621 and the outlet 622 of the valve body 620 is now open, and the gas within the respiratory system 1 can now exit via the valve 60, in the direction indicated by the arrows in FIG. 11. If the gas pressure is at or higher than the selected pressure level, and there is a further increase in gas pressure (e.g. from an increase in the flow rate of gases through the valve 60), even though the actuator 610 has already been lifted off the valve seat 623, F_{lift} will continue to displace the actuator 610 in an upward vertical direction. This will increase the gap between the actuator 610 and the valve seat 623 which, allow air to enter the valve body 620 and vent externally, and limit the increase in pressure to substantially maintain the gas pressure at the selected pressure level. At the same time, the lifting of the actuator 610 causes the biasing member 630 to compress further, which increases the variable resistance force generated by the biasing member 630, until it reaches an equilibrium position where F_{lift} equals F_{bias}, at which point the actuator 610 is not displaced any further away from the valve seat 623. The actuator 610 remains in that position to vent the gas flow externally, until the gas pressure changes again.

If there is a decrease in gas pressure (e.g. from a decrease in the flow rate of gases through the valve 60) and the gas pressure is still at or higher than the selected pressure level, F_{lift} is smaller than F_{bias}, so the net effect of the two forces will start moving the actuator 610 closer to the valve seat 623. This movement will reduce the size of the flow path within the valve 60 that allows gas flow to vent externally, to limit the decrease in pressure and substantially maintain the gas pressure at the selected pressure level. The actuator 610 remains in that position to vent the gas flow externally, until the gas pressure changes again.

As can be seen from the above, the operation of the valve 60 is different from a conventional pressure relief valve, which opens temporarily to reduce excessive pressure, and then remains closed after the excessive pressure is released. In comparison, the valve 60 of the present disclosure acts as a mechanical pressure regulation device, which assists with maintaining the gas pressure at the inlet of the valve 60 within a predetermined pressure range. More specifically, the pressure regulating effect is achieved by allowing the valve 60 to open, and stay open, as long as the pressure at the inlet of the valve 60 is above the predetermined pressure level. When used for infant resuscitation, the valve 60 is expected to stay open throughout PEEP delivery, and open to a greater extent (i.e. the actuator 610 is displaced further away from the valve seat 623) if the valve 60 is required to let a higher flow rate of gases pass through the valve 60.

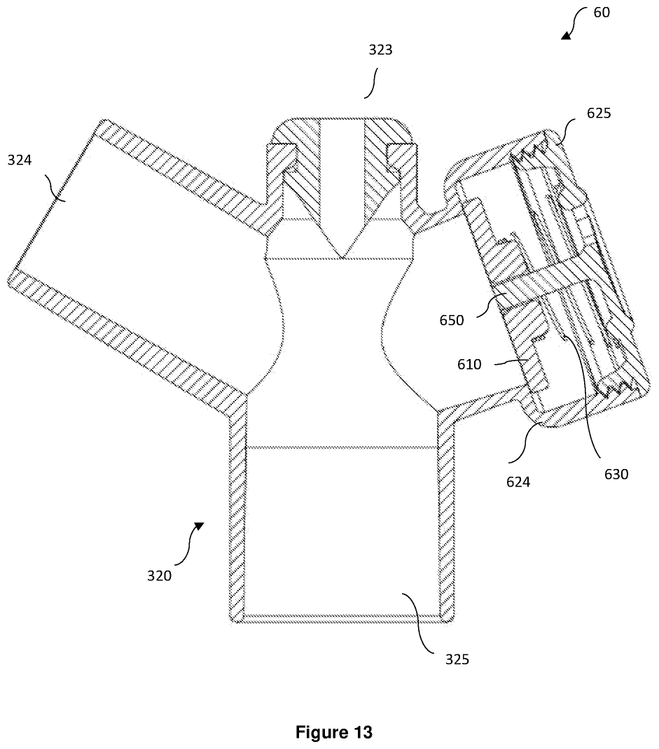

The valve 60 of the present disclosure may be used to regulate gas pressures delivered to a patient, in conjunction with a T-piece device 320. FIG. 12 shows an exploded perspective view of the valve 60 and a T-piece device 320. FIG. 13 shows a side cross sectional view of the valve 60 and the T piece device 320 when all the components are connected. In this embodiment, the housing 624 of the valve 60 may be integrally formed with the T-piece device 320. In other embodiments, the valve 60 may be detachably connected to the T-piece device 320, or another component of the respiratory system 1, via an adaptor as will be described further below.

The configuration of the T-piece device 320 is substantially similar to the device 320 shown in FIGS. 3 to 6. The T-piece device 320 comprises an inlet 324 arranged to receive a flow of breathable gas from a gas flow source, for example a respiratory apparatus 100. An outlet 325 of the device 320 is arranged to be connected to the patient interface (not shown) when delivering respiratory therapy. An optional valve, such as a duckbill valve 323 may be provided which can be used for insertion of an auxiliary equipment such as a catheter for fluid clearance or surfactant delivery, or a gas detection device, such as a CO₂ detector. The device 320 also comprises a vent, known as a PEEP vent, and the valve 60 is provided to the PEEP vent to act as a pressure regulating valve for the T-piece device 320.

The valve 60 and the T-piece device 320 may be used to regulate the gas pressures delivered to a patient, during bi-level resuscitation therapy. More specifically, the valve 60 and the T-piece device 320 may be used to regulate the PEEP pressure delivered to the patient, as follows.

As mentioned previously, when delivering bi-level resuscitation therapy, the gas pressure delivered to the patient is switched between PEEP and PIP in accordance with a desired breathing rate to be provided to the patient. After the initial calibration process, the respiratory system 1 may be arranged to enter into delivery mode, and provide a source gas flow to the patient at a substantially constant gas flow rate, as previously described in relation to FIG. 2. When used for regulating the PEEP pressure, the valve 60 may be configured such that the actuator 610 is lifted off the valve 623, and opens up the gas flow path between the inlet 621 and the outlet 622, when the pressure reaches a selected pressure level, wherein the selected pressure level is within the range of the PEEP pressure to be provided to the patient. For example, for an infant or neonate, the targeted PEEP pressure range may be around 5 cm H₂O, with a variation of −2 to +2 cm H₂O. That is, the predetermined and acceptable PEEP pressure range may be around 3 to 7 cm H₂O. In this case, the selected pressure level, at which point the actuator 610 is lifted the valve seat 623, may be set at 3.5 to 4.5 cm H₂O, which is within the predetermined PEEP pressure range. This ensures that valve 60 remains open during PEEP delivery, as long as the pressure is above the selected pressure level. It will be appreciated that the targeted PEEP pressure range may be within a range of 5 cm to 15 cm H₂O for an infant or neonatal patient, for example 6, 7, 8, 9, 10 cm H₂O.

The respiratory system 1 may be subject to flow variations caused by unintentional leaks or patient breathing (e.g. resulting in auto-PEEP) which often requires less or more gas flow to be vented externally via the valve 60. Patient breathing refers not just to spontaneous patient breathing but also to other inspiratory and expiratory flows of the patient e.g. during assisted ventilation which could potentially cause a pressure variation within the respiratory system 1. In the event of an unintentional leak, the pressure within the respiratory system 1 may decrease, which reduces the lifting force exerted onto the actuator 610 in the upward direction. Accordingly, the actuator 610 may be moved closer to the valve seat 623, to reduce the size of the gas flow path between the inlet and the outlet of the valve. This will limit the decrease in pressure such that pressure is maintained at substantially the selected pressure level (within the predetermined PEEP pressure range). Alternatively, if the pressure drops to a level which is below the selected pressure of 3.5 to 4.5 cm H₂O, the actuator 610 may be seated on the valve seat 623 as shown in FIG. 10, to close off the valve 60 completely.

If the patient exhales during PEEP delivery, there is likely to be an increased gas flow in the respiratory system caused by the patient, as the respiratory system 1 is set to deliver a fixed source gas flow, particularly when the respiratory system 1 of FIG. 2 is used. The increased gas flow may elevate the gas pressure in the system, and increases the lifting force in the upward direction. Although the actuator 610 has already been lifted off the valve 623, this increase in pressure will displace the actuator 610 further away from the valve 623, thereby increasing the size of the flow path between the inlet 621 and the outlet 622 of the valve, and letting the additional gas flow to vent through the valve 60. In this way, the valve 60 is able to limit the increase in pressure to regulate the gas pressure such that it remains within the predetermined PEEP pressure range.

During PIP delivery, the outlet 622 of the valve 60 is arranged to be occluded, for example by a finger of an operator, to effectively close off the valve 60, regardless of the gas pressure within the respiratory system 1. When PIP delivery finishes, the occlusion may be removed from the outlet 622, allowing the valve 60 to regulate PEEP pressure again as described above.

The configuration of the biasing member 630, the exposed area of the actuator 610, and the inlet of the valve 60 should be carefully determined, and mutually adapted with respect to each other, such that the valve 60 is able to regulate the gas pressure when required and maintains the gas pressure of the respiratory system 1 within the predetermined PEEP pressure range.

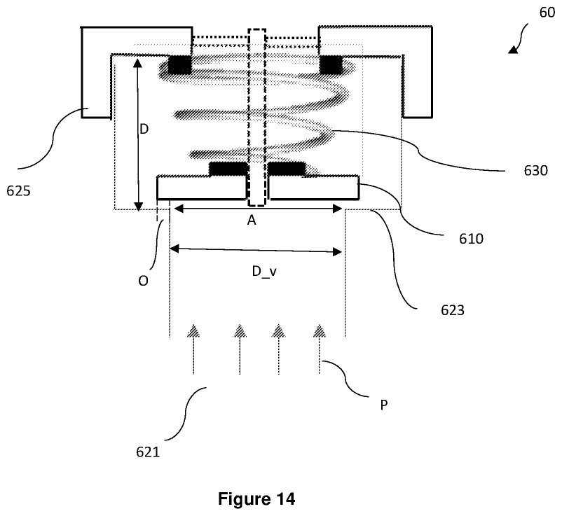

FIG. 14 schematically represents the structural components that are in the valve 60. As mentioned previously, the pressure regulating mechanism of the valve 60 is determined by the relative strengths of F_{bias} and F_{lift}. Accordingly, the key parameters that influence the pressure regulating mechanism of the valve 60 are: the gas pressure at the inlet 621 (P), area of the actuator 610 that is exposed to the gas pressure at the valve inlet (A), initial compression of the biasing member (x_initialcompession), depth of the valve body 620 (which determines a potential maximum x_lift), and the spring constant (k). The spring constant of the biasing member 630 is also correlated with other parameters of the biasing member 630, such as number of coils, diameter of the coils, diameter of the coil wire, spring pitch, and similar thereof.

Although the example above uses bi-level resuscitation therapy as an example, and the valve 60 is configured to regulate PEEP delivery pressure, it should be appreciated that the valve 60 may also be adapted such that is regulates another pressure range, by selecting different parameters for the biasing member 630 and the exposed area of the actuator 610. As mentioned above, the pressure above which the actuator 610 is lifted off the valve seat 623, can be calculated from P=k*x_initialcompression/A. That is, once the pressure P is selected, or known, other parameters of the valve may be derived using this equation.

In addition to selecting a suitable spring constant (k), initial compression of the spring (x_initialcompression), and the exposed area of the actuator 610 (A), there are other design considerations which contribute to how the valve 60 may be configured, at least some of which are set out below.

Gas flow rate via the valve 60: to regulate PEEP pressure, the gas flow rate that the valve 60 is able to regulate should be within a range of 5 to 15 L/min when used with infants. For adult patients, the gas flow rate may be within a range of 1 to 150 L/min, or within a range of 20 to 70 L/min, or up to 50 L/min, or up to 30 L/min. This range may be approximately the same as the source flow rate which the respiratory system 1 is set to provide to a patient.

Predetermined range of PEEP pressure: for infant resuscitation, the targeted PEEP pressure is usually within a range of 4 to 15 cm H₂O. The valve 60 should be configured such that it is able to regulate the gas pressure within the respiratory system 1, such that it stays within this range. In some embodiments, the valve 60 is configured to regulate the pressure of the breathable gas within the respiratory system 1 by a variation of −2 to +2 cm H₂O, −1 to +1 cm H₂O, or by a variation of −0.5 to +0.5 cm H₂O. That is, if the respiratory system 1 is set to deliver PEEP to a patient at a targeted PEEP pressure of 5 cm H₂O, at a given flow rate, then the valve 60 is configured to regulate the PEEP pressure such that it stays within a range of 3 to 7 cm H₂O, or 4 to 6 cm H₂O, or 4.5 to 5.5 cm H₂O, at that given flow rate, regardless of any flow variations experienced by the system.

Depth of the valve: the depth of the valve 60 (D) at least in part determines the initial compressed length of the biasing member 630 (x_initialcompression), and a maximum x_lift that is able to be achieved. In at least one embodiment, the depth of the valve is approximately 3 to 6 mm.

Configuration of the biasing member 630: configuration of the biasing member includes selection of suitable spring constant, spring wire diameter, sizes of the spring coils, number of the spring coils, spring pitch, and so on. In at least one embodiment, the spring constant is less than 0.05 N/mm. Preferably, the spring constant is within a range of 0.005 to 0.02 N/mm.

Actuator: The exposed area of the actuator 610 (A) and the dimension of the valve inlet (D_v) 621 may be selected so that the pressure to open the valve 60, and the pressure that the valve regulates are similar. In at least some embodiments, the cross sectional area of the actuator 610, or the cross sectional area of the exposed area of the actuator 610 (A) is between 50 to 320 mm². In other embodiments, the cross sectional area of the actuator 610, or the cross sectional rea of the exposed area of the actuator 610 may be within a range of 100 to 250 mm², or within a range of 120 to 200 mm², or within a range of 140 to 180 mm². In one embodiment, when the actuator 610 is in the seated position, the exposed area (A) may be, for example about 160mm². In some embodiments, the actuator 610 may be made from a relatively rigid material, such as polyurethane, so as to reduce the likelihood of flexing of the actuator 610. However other materials, for example, thermoplastic elastomer materials such as silicon, or rubber may also be suitable.

Further, the exposed area of the actuator 610, and the spring constant, are mutually selected such that a relatively small displacement of the actuator 610 (i.e. in mm range, or a fraction of a millimetre) with respect to the valve seat 623 is sufficient to allow the valve 60 to achieve its pressure regulation effect. That is, this relationship between the exposed area of the actuator 610 and the spring constant of the biasing member 630 determines the pressure regulation effect described above. Due to this relationship, increases and decreases in pressure (respectively resulting from increases and decreases in the flow rate of gases through the device 320 and valve 60, as described above) are limited by the relatively small displacement of the actuator 610 which occurs, such that gas pressure is regulated to be maintained at the selected pressure level (e.g. within the predetermined target PEEP pressure range).

As a relatively small displacement of the actuator 610 may be sufficient to allow the valve 60 to achieve its pressure regulation effect, the actuator orifice may be formed as a blind bore (i.e. instead of a through hole), which receives a lower end of the shaft 650.

The cross sectional area of the actuator 610 is preferably smaller, or considerably smaller than an inner transverse dimension of the valve body 620, so there is no significant additional resistance to flow when gases pass between an internal side wall of the valve body 620 and the actuator 610. The resistance to flow through the valve 60 is predominantly determined by the gap formed between the actuator 610, and the valve seat 623. The resistance to flow through the valve 60 is predominantly determined by the displacement of the actuator 610 with respect to the valve seat 623. Generally, for a given flow rate of gases through the valve 60, an increase in the resistance to flow through the valve 60 results in an increase in pressure and contrarily, a decrease in the resistance to flow through the valve 60 results in a decrease in pressure.

Flow consistency: The inlet 324 of the T-piece device 320 may have the same size as the inlet 621 of the valve 60, to maintain a consistent flow between the inlet 324 of the T-piece device, and the inlet 621 of the valve 60, when the valve 60 is open.

Overlap of the actuator 610 and the valve seat 623: the overlap between the actuator 610 and the valve seat 623 (O) is preferably kept small, to avoid potential “sticking” of the components and to avoid creating additional restriction to flow. In at least one embodiment, the overlap O is within a range of 0.2 mm to 3 mm, or within a range of 0.2 mm to 2 mm, in a radial direction.

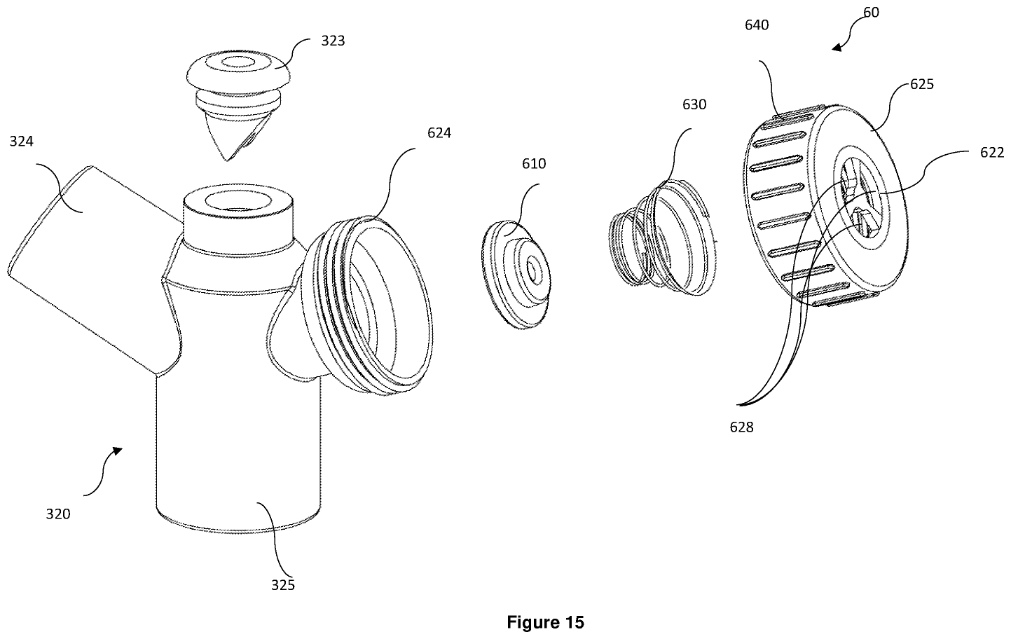

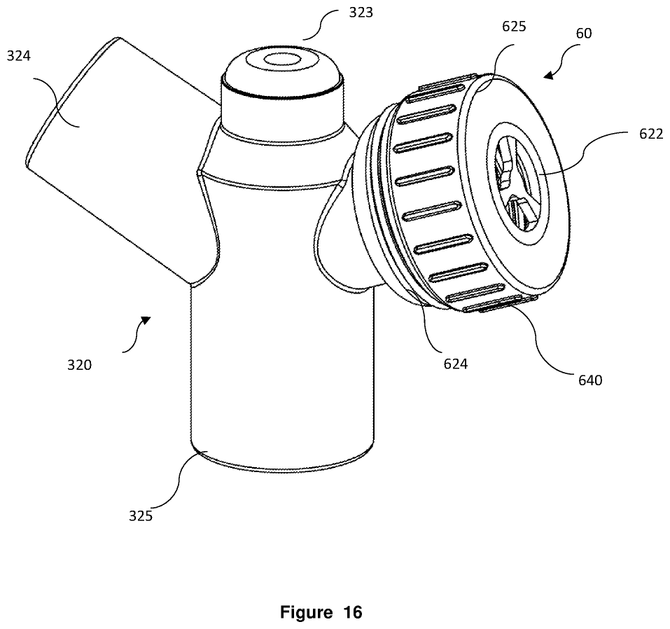

FIGS. 15 and 16 show another embodiment of a valve 60 according to the present disclosure. The configuration of the valve 60 is substantially similar to the embodiment illustrated in FIGS. 10, 11, with a few modifications. FIG. 15 shows an exploded view of the components of the valve 60. The valve body 620 again includes a housing 624 and an outlet member 625, which are coupled to each other via a threaded coupling. Threaded portions are now formed on an external wall of the housing 624, and corresponding threaded portions are formed on an internal wall of the outlet member 625. After the valve 60 is connected to a T-piece device 320, the housing 624 may extend partially into the outlet member 625.

Gripping formations 640 are formed on an exterior surface of the outlet member 625. The gripping formations 640 may include an array of parallel ridges which are evenly distributed around the exterior surface of the outlet member 625. The gripping formations 640 may facilitate easier connection and disconnection of the valve 60 with the T-piece device 320. The outlet member 625 may also be utilised to adjust an initial compression of the biasing member 630, by twisting the outlet member 625 with respect to the housing 624. For example, the outlet member 625 may be twisted to increase the height of the internal space created by the housing 624 and the outlet member 625, thereby reducing the initial compression of the biasing member 630. Alternatively, the outlet member 625 may be twisted to decrease this height, causing the biasing member 630 to be compressed further. By adjusting the initial compression of the biasing member 630, the targeted pressure range which the valve 60 is arranged to regulate is also adjusted. As mentioned above, the selected pressure level at which the valve 60 opens, is determined by P=k*x_initialcompression/A. The valve 60 opens once P is exceeded. When the biasing member 630 has a greater initial compression (by reducing the height of the internal space created by the housing 624 and the outlet member 625), the selected pressure level at which the valve 60 opens, is also higher. Similarly, when the biasing member 630 has a smaller initial compression, the selected pressure level at which the valve 60 opens is lowered as well.

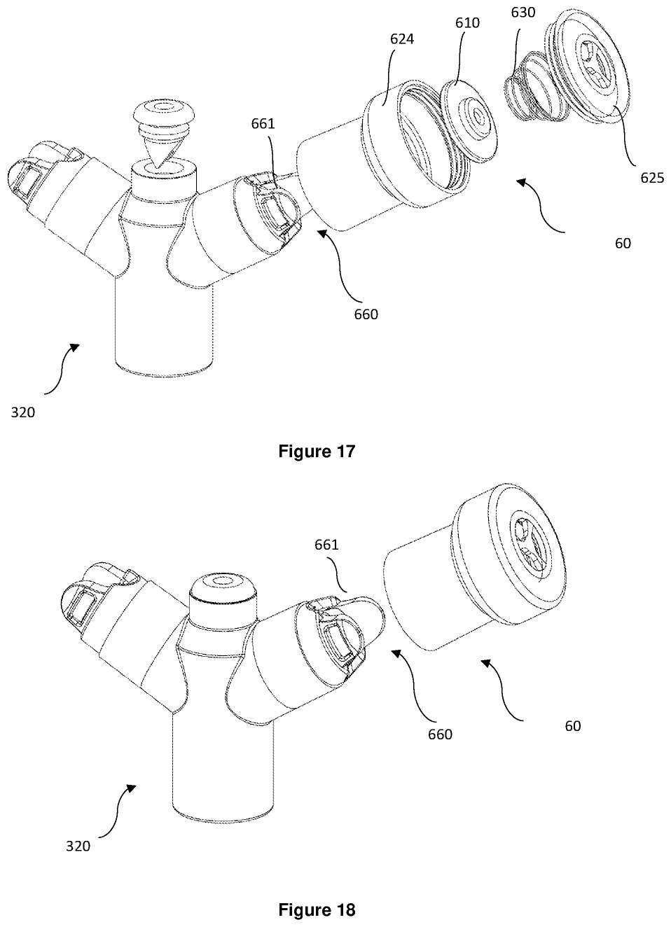

FIGS. 17, 18, 19, 20, 21 show various different views of another embodiment of a valve 60. In previous embodiments described above, the valve 60 may be at least partially integrally formed with a T-piece device 320. FIGS. 17-21 illustrate another embodiment of a valve 60 which can be detachably connected to a T-piece device 320, or another venting orifice of the respiratory system as will be described further below.

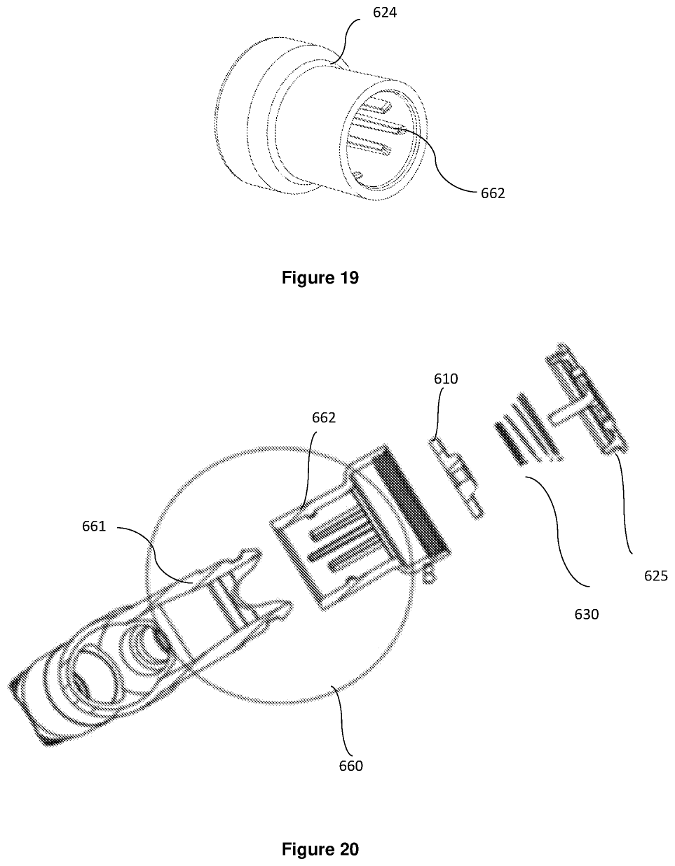

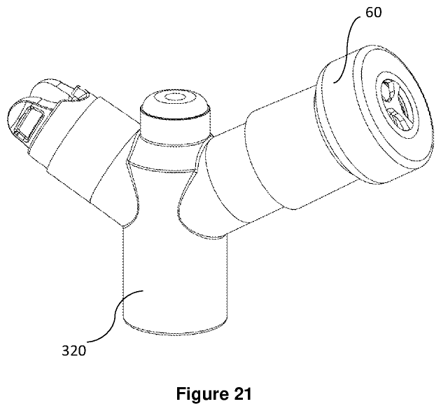

In this embodiment, the valve 60 includes an adaptor 660, allowing the valve 60 to be detached and reattached to other locations of a respiratory system 1. Examples of such adaptors are described in U.S. Pat. No. 9,808,612B2, the content of which is incorporated herein in its entirety. The adaptor 660 includes male and female connector portions 661 and 662, configured to engage with each other in order to form a detachable connection. The male connector portions 661 includes a pair of locking fingers and are arranged to be received by the female connector portions 662. The female and male connector portions 662, 661 may be provided to the valve 60, and a PEEP vent 322 of a T-piece device 320 respectively to facilitate an easier coupling between the two components. Or alternatively, the male connector portion 661 may be provided to the valve 60, and the female connector portion 662 may be provided to the PEEP vent 322. FIG. 19 shows that the female connector portions 662 may be formed on an internal wall of the housing 624. The adaptor 660 allows the valve 60 to be connected to the PEEP vent of the device 320, by simply pushing the housing 624 of the valve 60 toward the PEEP vent 322, until the male and female connector portions 661 and 662 engage with each other and form a connection. FIG. 21 shows the valve 60 being coupled to the device 320, through this coupling mechanism. To detach the valve 60 from the device 320, a user may pull the valve 60 away from the device 320, which causes the female and male connector portions 662, 661 to disengage from each other.

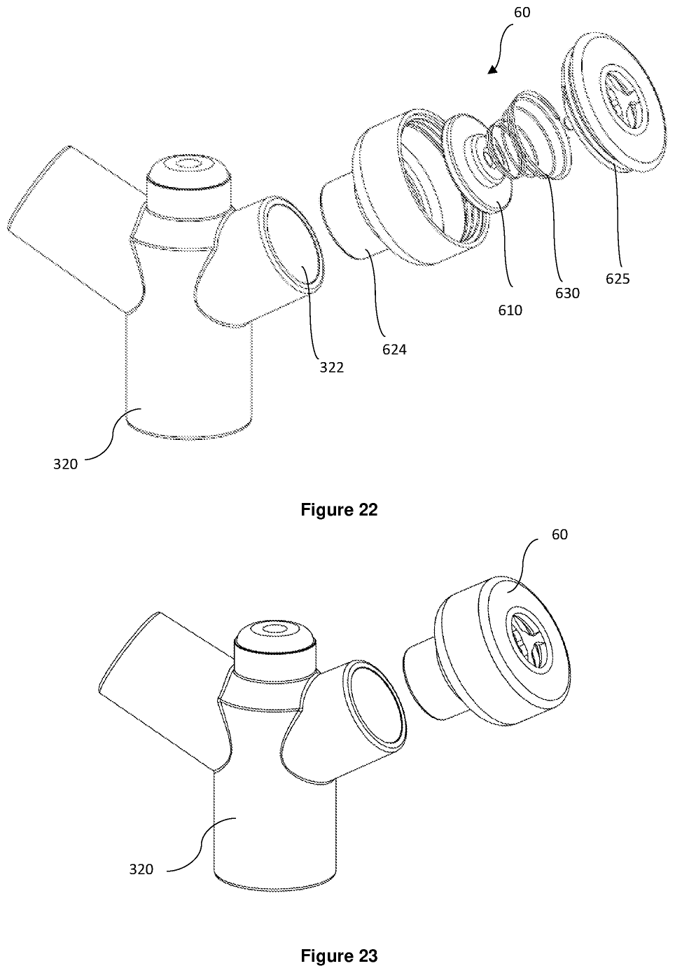

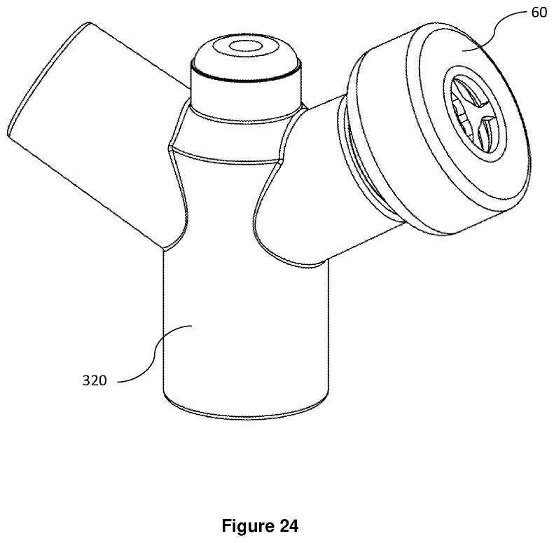

FIGS. 22-24 illustrate yet another embodiment of a valve 60 configured to connect to a PEEP vent of a T-piece device 320. To facilitate easier connection of the valve 60 and the T-piece device 320, the housing 624 may be configured to have a tapering profile, enabling an end of the housing 624 to be directly inserted into the PEEP vent of the T-piece device 320 in order to form a connection.

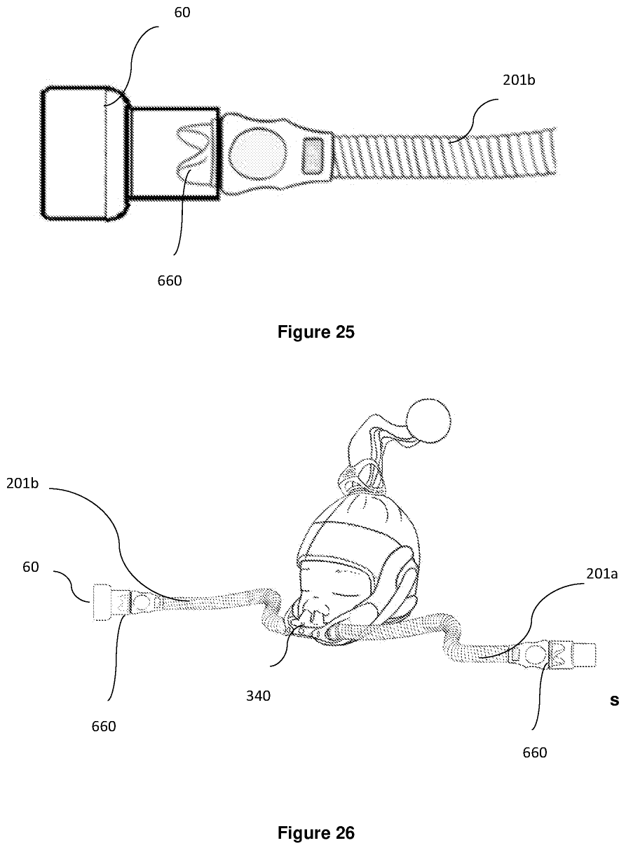

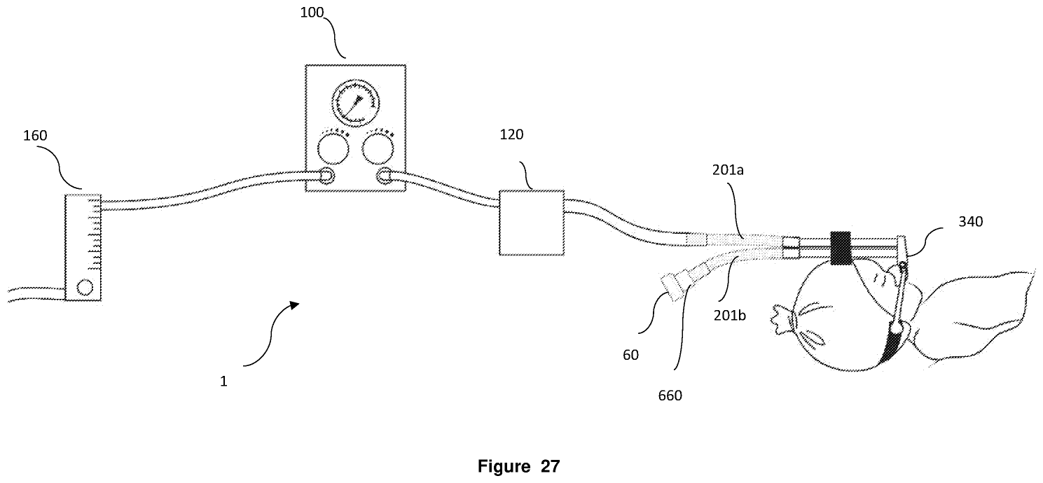

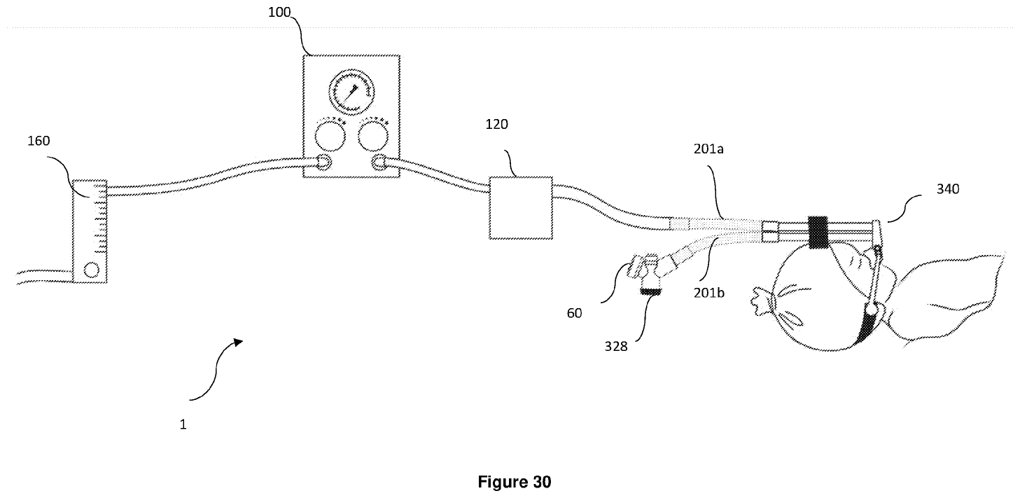

The detachable connection mechanisms described above may also be used to reduce set-up complexity and to enable resuscitation therapy to be provided through a different interface, for example through an infant CPAP interface. Examples of infant CPAP interfaces have been described in WO2021176338A1, the content of which is incorporated herein in its entirety. In some CPAP respiratory systems, a flow of expiratory gases is directed from a patient interface via an expiratory conduit to a resistance device, such as a bubbler device. The valve 60 of the present disclosure may be used to act a resistance device in such CPAP respiratory devices to replace the previous bubbler devices. The valve 60 may be beneficial in CPAP respiratory systems as the valve 60 can maintain a consistent CPAP pressure whilst the patient is spontaneous breathing (which results in changes in the flow rate of gases through the valve during inspiration and expiration). FIGS. 25-27, 28-30 illustrate examples of using a valve 60 of the present disclosure with infant CPAP interfaces, to deliver resuscitation therapy to a patient.

FIGS. 25-27 show an example CPAP respiratory system 1 in which a valve 60 of the present disclosure is used. The valve 60 may include a detachable connector 660 as previously described with reference to FIGS. 19-29. In the illustrated arrangement, the patient interface 340 receives an inspiratory flow of gases via an inspiratory conduit 201a. A flow of the expiratory gases can be directed from the interface 340 via an expiratory conduit 201b to a variable flow resistance device, which in the illustrated arrangement is a valve 60. An optional humidifier system 120 is provided to humidify the inspiratory flow of gases. The humidified inspiratory flow of gases is delivered to the airway of the patient by inspiratory conduit 201a and the patient interface 340. Excess and expired gases are evacuated from the patient interface 340 by the expiratory conduit 201b. The valve 60 provides a variable flow resistance to the expiratory flow of the gases exiting the system 1 to provide a desirable patient pressure. In at least one embodiment, the valve 60 and the respiratory system 1 may be configured to provide bi-level resuscitation therapy as previously described. In use, the outlet of the valve 60 may be occluded, such that the respiratory system 1 delivers PIP to the patient. As the occlusion is removed, the respiratory system 1 delivers PEEP to the patient, and the valve 60 regulates the gas pressure delivered to the patient such that it remains within the predetermined PEEP range.

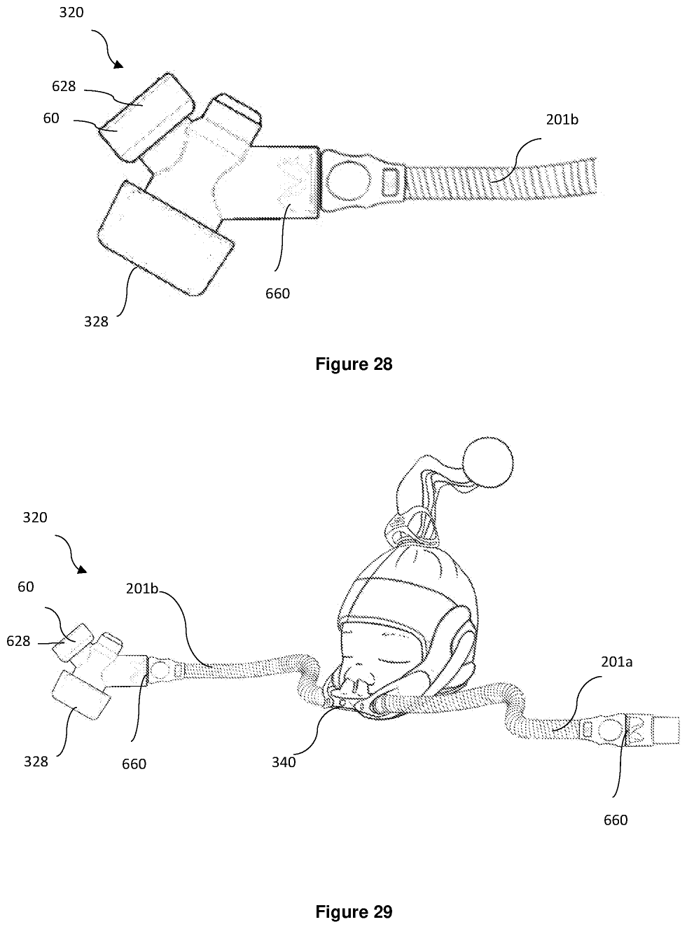

FIGS. 28-30 illustrate another example of using a valve 60 of the present disclosure with a CPAP respiratory system 1, in combination with a T-piece device 320. With reference to FIG. 28, an adaptor 660 (female or male) may be provided to an inlet of the T-piece device 320, and a complimentary adaptor 660 may be provided to the expiratory conduit 201b of the CPAP interface 340. This will allow a quick connection to be made between the T-piece 320 and the CPAP interface 340. In addition, another adaptor 660 may be provided to the PEEP vent of the T-piece device 320, and the valve 60, to enable the valve 60 to be detachably connected to the T-piece device 320 as in previous embodiments. The outlet of the T-piece 320 is then covered by a protective cover such that all of the expiratory flow of gases pass through the valve 60. This configuration may also be used to provide resuscitation therapy to the patient, for example, by occluding the outlet 622 of the valve 60 with a finger or a digit, to switch between PIP and PEEP.

### EXAMPLES

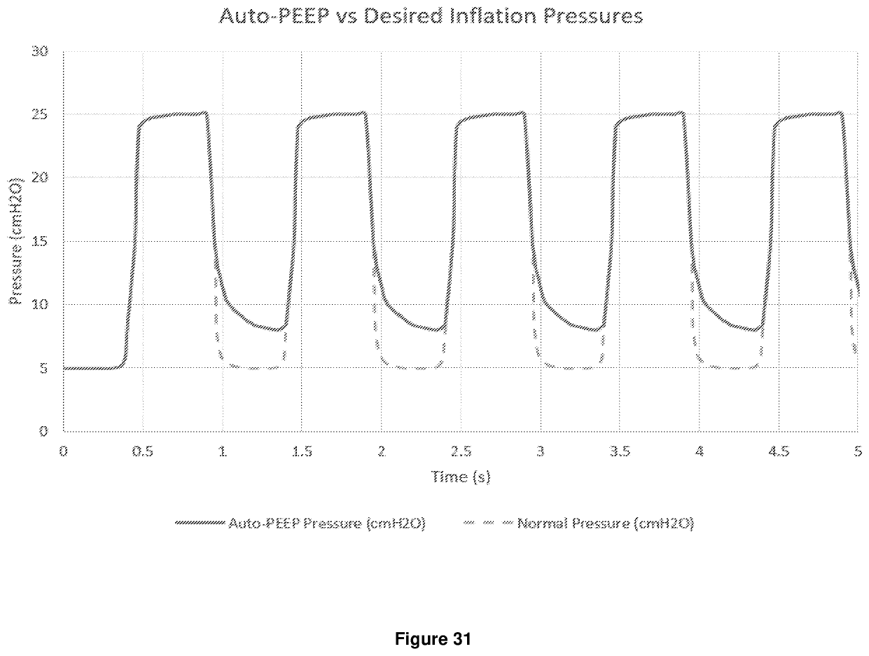

In prior art systems, when there is excessive gas in the respiratory system 1 caused by patient's breathing, the gas pressure at the inlet of prior PEEP valve may increase, and lead to auto-PEEP. FIG. 31 demonstrates the effect of auto-PEEP and its influence on the gas pressure in previous respiratory systems, i.e. without relying on the valve 60 of the present disclosure to perform pressure regulation to maintain gas pressure within the selected pressure when there is an increase in the flow rate of gases through the valve 60. Two pressure curves are shown in FIG. 31: an auto-PEEP pressure curve, and a targeted pressure curve showing the level of pressures should be achieved during respiratory therapy. As can be seen in FIG. 31, during PEEP delivery, the targeted pressure to be provided to the patient may be set at 5 cm H₂O, when the respiratory apparatus is set to deliver a constant gas flow rate of 8 L/min. If, at the end of an expiratory cycle, there is still an expiratory flow of 2 L/min from the patient, then the total flow that is required to be released will be 10 L/min. If a fixed flow restriction device (e.g. the previous PEEP valve as illustrated in FIG. 8) is used to vent gas externally, the increased flow rate of 10 L/min is likely to have an effect of elevating the pressure within the respiratory system temporarily to approximately 8.3 cm H₂O. Thus, rather than allowing the gas pressure to reduce to the desired level of 5 cm H₂O, the actual pressure delivered to the patient is higher in the event of auto-PEEP.

The valve 60 of the present disclosure is configured to prevent or reduce the increase of pressure, due to expiratory flow, which prevents or reduces the event of auto-PEEP occurring. If the pressure at the inlet 621 of the valve 60 is below the selected pressure level at which the valve 60 should be open, then the actuator 610 remains seated on the valve seat 623. As the gas pressure meets or exceeds the selected pressure level, the actuator 610 is lifted off the valve 623, thereby allowing gas to flow through the valve 60 and vent externally. In the event of auto-PEEP, due to additional gas flow (an increase in the flow rate of gases through the valve 60) caused by patient's breathing, there is likely be to an increase in the gas pressure at the inlet 621 of the valve 60. This increase in gas pressure is translated into an increased lifting force applied onto the actuator 610, and causes the actuator 610 to be displaced further away from the valve seat 623, such that the gas flow path between the inlet and the outlet of the valve 60 is increased in size. As described above, the displacement of the actuator 610 from the valve seat 623 is such that the increase in the gas pressure is limited to regulate the gas pressure and maintain the gas pressure at the selected pressure level (e.g. within the predetermined PEEP pressure range).

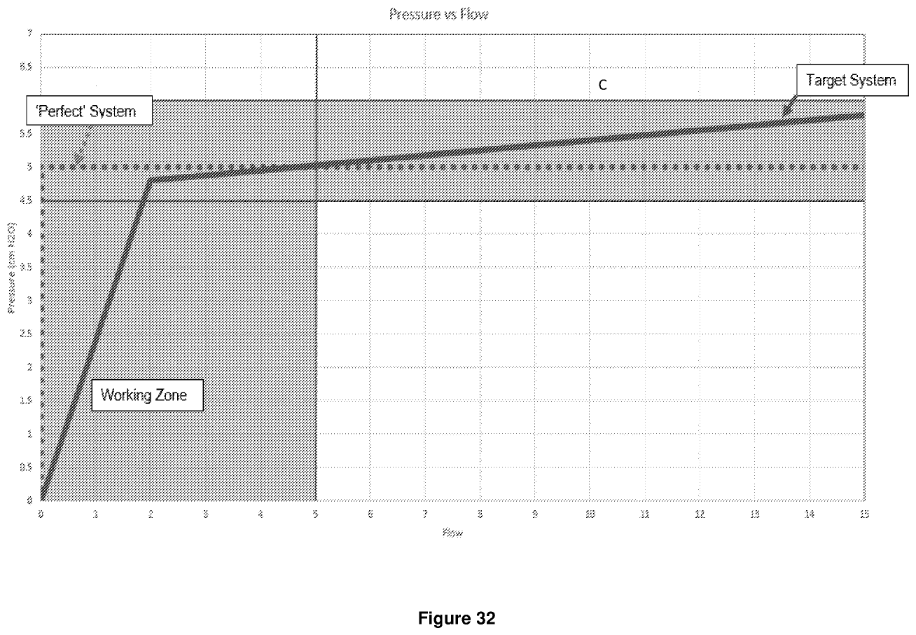

FIG. 32 illustrates a pressure curve generated by a “perfect” system, where the PEEP pressure remains at 5 cm H₂O at any flow rate. The PEEP pressure regulated by the valve 60 is shown as a comparison. The solid line demonstrates that the system is still able to deliver more than 4.5 cmH₂O for flows above 2 L/min, with the pressure very slowly increasing as flow increases, crossing 5 cmH₂O at 5 L/min, and approaching 5.8 cmH₂O at 15 L/min. Even though the flow rate has increased from 2 L/min to 15 L/min, the corresponding PEEP pressure variation is only from 4.5 cmH₂O to 5.8 cmH₂O. The shaded area represents the ‘working zone’ where any point within this area is an acceptable PEEP pressure at that flow rate. FIG. 32 shows that the valve 60 limits any increases or decreases in gas pressure (respectively resulting from increases or decreases in the flow rate of gases, through the device 320 and valve 60, of between about 2 L/min and about 15 L/min,) such that gas pressure is regulated to be maintained at the selected pressure level (within the predetermined target PEEP pressure range).

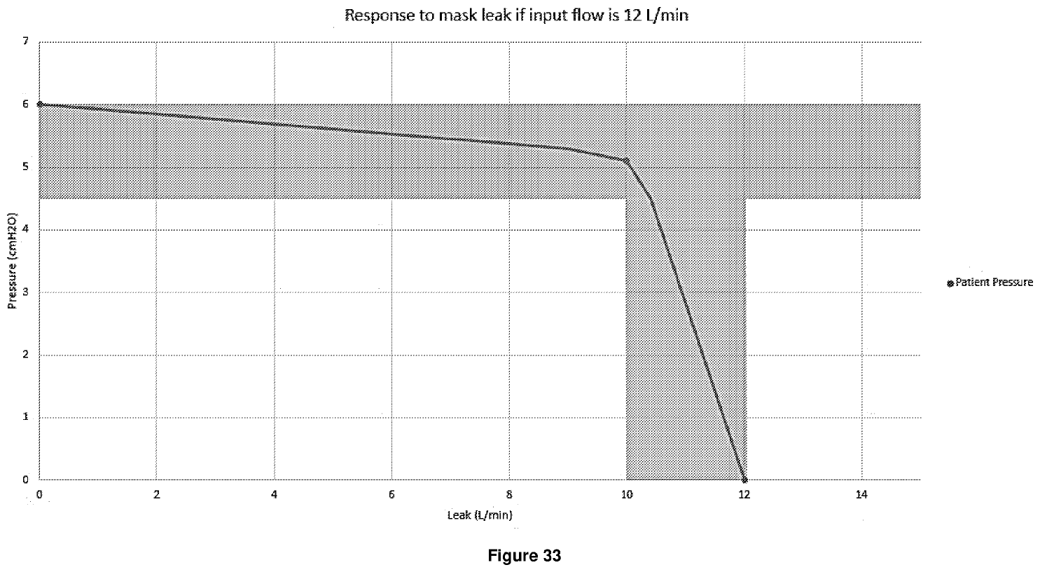

FIG. 33 shows the response to leak of the valve 60 at an input flow rate of up to 12 L/min. In the event of an unintentional leak (a decrease in the flow rate of gases through the valve 60), to the extent that it causes a drop in the gas pressure at the inlet 621, the size of the gas flow path between the inlet and the outlet of the valve 60 is reduced, to compensate for the pressure loss. This decrease in gas pressure is translated into a decreased lifting force applied onto the actuator 610, and causes the actuator 610 to be displaced closer to the valve seat 623, such that the gas flow path between the inlet and the outlet of the valve 60 is decreased in size. As described above, the displacement of the actuator 610 from the valve seat 623 is such that the decrease in the gas pressure is limited to regulate the gas pressure and maintain the gas pressure at the selected pressure level (e.g. within the predetermined PEEP pressure range). If the leak continues to increase, to a level greater than around 10.5-11 L/min when 12 L/min is being provided, causing further pressure drop in the respiratory system 1, the actuator 621 may return to its valve seat 623 and prevent gas flow through the valve 60.

In FIG. 32, as flow rate is increased from 2 to 15 L/min, the valve 60 is required to displace its actuator 623 further away from the valve seat 623, allowing the higher flow rate of gas through the valve 60 whist ensuring the gas pressure stays with the PEEP pressure range. In comparison, when there is a leak, the gas flow rate through the valve 60 reduces, whist still maintaining the pressure within the PEEP pressure range. As illustrated in FIG. 33, when the level of leak increases from 0 L/min to about 10.5 L/min, at an input flow rate of 12 L/min, the delivered PEEP pressure only reduces slightly from 6 cm H₂O to 4.5 cm H₂O and is maintained at the selected pressure level within the predetermined PEEP pressure range. The shaded area represents the ‘working zone’ (predetermined PEEP pressure range) where any point within this area is an acceptable PEEP pressure at that input flow rate.

Positive End Expiratory Pressure (PEEP) is also known as Peak End Expiratory Pressure and the two terms are often used interchangeably in the context of respiratory therapy systems and methods.

In this specification, adjectives such as left and right, top and bottom, hot and cold, first and second, and the like may be used to distinguish one element or action from another element or action without necessarily requiring or implying any actual such relationship or order. Where context permits, reference to a component, an integer or step (or the alike) is not to be construed as being limited to only one of that component, integer, or step, but rather could be one or more of that component, integer or step.

In this specification, the terms ‘comprises’, ‘comprising’, ‘includes’, ‘including’, or similar terms are intended to mean a non-exclusive inclusion, such that a method, system or apparatus that comprises a list of elements does not include those elements solely, but may well include other elements not listed.

The above description relating to embodiments of the present disclosure is provided for purposes of description to one of ordinary skill in the related art. It is not intended to be exhaustive or to limit the disclosure to a single disclosed embodiment. As mentioned above, numerous alternatives and variations to the present disclosure will be apparent to those skilled in the art from the above teaching. Accordingly, while some alternative embodiments have been discussed specifically, other embodiments will be apparent or relatively easily developed by those of ordinary skill in the art. The present disclosure is intended to embrace all modifications, alternatives, and variations that have been discussed herein, and other embodiments that fall within the spirit and scope of the above description.

1. A valve for use with a respiratory system arranged to convey a breathable gas to a patient, wherein the valve allows gas from within the respiratory system to exit, comprising:
a valve body including an inlet and an outlet, said inlet configured to be in fluid communication with the respiratory system;
an actuator disposed within the valve body, in a flow path between the inlet and the outlet,
wherein the actuator is movable by the gas, movement of the actuator being at least partially dependent on a pressure of the gas at the inlet, wherein said movement adjusts the flow path between the inlet and the outlet, to regulate the pressure of the gas in the respiratory system within a predetermined range.

2. The valve of claim 1, wherein the actuator is biased towards the inlet of the valve.

3. A valve for use with a respiratory system arranged to convey a breathable gas to a patient, wherein the valve allows gas from within the respiratory system to exit, comprising:
a valve body including an inlet and an outlet, said inlet configured to be in fluid communication with the respiratory system;
an actuator disposed within the valve body, in a flow path between the inlet and the outlet,
wherein the actuator is biased towards the inlet and movement of the actuator away from the inlet being at least partially dependent on a pressure of the gas at the inlet, said movement adjusting the flow path between the inlet and the outlet, to regulate the pressure of the gas in the respiratory system within a predetermined range.

4. The valve of claim 1 or 3, wherein the actuator is caused to move away from the inlet when the pressure exceeds a selected pressure level.

5. The valve of any one of preceding claims, wherein the predetermined range is a predetermined PEEP pressure range.

6. The valve of claim 5, wherein the selected pressure level is within the predetermined PEEP pressure range.

7. A valve for use with a respiratory system arranged to convey a breathable gas to a patient, wherein the valve allows gas from within the respiratory system to exit, comprising:
a valve body including an inlet and an outlet, said inlet configured to be in fluid communication with the respiratory system;
an actuator disposed within the valve body, in a flow path between the inlet and the outlet,
wherein the actuator is biased towards the inlet, the actuator being caused to move away from the inlet when the pressure exceeds a selected pressure level, said movement adjusting the flow path between the inlet and the outlet, to regulate the pressure of the gas in the respiratory system within a predetermined PEEP pressure range, wherein the selected pressure level is within the predetermined PEEP pressure range.

8. The valve of any one of preceding claims, where the valve comprises a biasing member, operatively coupled to the actuator.

9. The valve of claim 8, wherein the actuator is biased towards the inlet by the biasing member.

10. The valve of claim 8 or 9, wherein the biasing member applies a variable resistance force onto the actuator during movement thereof.

11. The valve of claim 10, wherein the variable resistance force applied by the biasing member counters a force exerted onto the actuator caused by the gas pressure at the inlet.

12. The valve of any one of preceding claims, wherein the flow path between the inlet and the outlet of the valve is closed off, when the pressure at the inlet is below the selected pressure level.

13. The valve of claim 12, wherein when the pressure at the inlet is above the selected pressure level, it overcomes the variable resistance force applied onto the actuator by the biasing member, and opens up the flow path between the inlet and the outlet of the valve.

14. The valve of any one of claims 8 to 13, wherein the biasing member biases the actuator against a portion of the valve body when the flow path is closed off.

15. The valve of claim 14, wherein the portion of the valve body forms a valve seat for the actuator.

16. The valve of claim 15, wherein the flow path between the inlet and the outlet of the valve is opened up when the actuator is displaced from the valve seat.

17. The valve of claim 16, wherein the flow path is at least in part determined by a relative displacement of the actuator from the valve seat.

18. The valve of claim 17, wherein the flow path increases its size as the actuator is displaced further away from the valve seat.

19. The valve of claim 17 or 18, wherein the flow path decreases its size as the actuator returns to the valve seat.

20. The valve of any one of preceding claims, wherein the valve includes a supporting member for the actuator, for guiding and stabilising the movement of the actuator.

21. The valve of claim 20, wherein the supporting member is an elongate shaft, along which the actuator is arranged to slide during its movement.

22. The valve of claim 21, wherein the actuator includes an orifice, for receiving the shaft therein.

23. The valve of claim 22, wherein the orifice and a transverse cross section of the shaft are configured to have a substantially similar shape and/or size.

24. The valve of claim 22, wherein the orifice has a substantially square shape, and the shaft has a circular transverse cross section.

25. The valve of any one of claims 22 to 24, wherein the orifice has a diameter of approximately 1 to 5 mm.

26. The valve of any one of preceding claims, wherein the actuator comprises a substantially planar lower surface, or, the actuator comprises a circular disk, wherein the circular disk comprises a substantially planar lower surface.

27. The valve of claim 26, wherein the dimension, or an area of the circular disk closely matches the dimension of the valve inlet.

28. The valve of any one of preceding claims, wherein the actuator has an area greater than a cross sectional area of the inlet, preferably a difference between the area of the actuator and the inlet of the valve body is within a range of 0 to 80 mm², or 1 to 20 mm².

29. The valve of any one of claims 8 to 28, wherein the actuator comprises a portion for engaging the biasing member.

30. The valve of any one of preceding claims, wherein the valve body comprises a housing, and an outlet member.

31. The valve of claim 30, wherein the outlet member is arranged to couple to an end of the housing.

32. The valve of claim 30 or 31, wherein the housing comprises a hollow cylindrical shape, and the valve seat is formed on an interior wall of the housing.

33. The valve of any one of claims 30 to 32, wherein the outlet member comprises at least one outlet member orifice, to vent the gas received at the inlet of the valve to ambient air.

34. The valve of claim 33, wherein the outlet member orifice has a minimum area of approximately 20 mm².

35. The valve of any one of preceding claims, wherein the outlet of the valve is arranged to be occluded, when delivering PIP to the patient.

36. The valve of any one of preceding claims, wherein the outlet of the valve is arranged to be occluded by a finger or a digit of an operator during PIP delivery.

37. The valve of any one of preceding claims, wherein the outlet of the valve is not occluded, when delivering PEEP to the patient.

38. The valve of any one of claims 22 to 37, wherein the shaft is received by the orifice of the actuator at a first end, and connected to a portion of the valve body at a second end.

39. The vale of any one of claims 8 to 38, wherein the biasing member is a spring, a coil spring, or a conical coil spring.

40. The valve of any one of claims 8 to 39, wherein the biasing member is held in a compressed state, when the actuator is biased toward the valve seat, and is compressed further, as the actuator is lifted off the valve seat.

41. The valve of claim 10, wherein the variable resistance applied onto the actuator by the biasing member is at least partially dependent on: spring constant, compression of the spring, and/or displacement of the actuator from the valve seat.

42. The valve of claim 40, wherein the variable resistance applied onto the actuator by the biasing member may be calculated from F_{bias}=k*(x_initialCompression+x_lift), where
• k=spring constant of the spring;
• x_initial compression=initial compressed length of the spring, when the flow path is closed off (i.e. a difference between the original uncompressed length of the spring, and the length of the spring after it has been initially compressed and placed within the valve body);
• x_lift=displacement of the actuator from the valve seat, at pressure P.

43. The valve of claim 10, 41 or 42, wherein gas pressure at the inlet applies a lifting force (F_{lift}) onto the actuator, in a direction which is opposite to the variable resistance applied onto the actuator by the biasing member.

44. The valve of claim 43, wherein the lifting force may be calculated from F_{lift}=P*A_{actuator}, where
• P=gas pressure at the valve inlet;
• A_{actuator}=area of the actuator exposed to the gas pressure at the valve inlet.

45. The valve of claim 44, wherein the position of the actuator is determined by the relative relationship of F_{bias} and F_{lift}.

46. The valve of claim 45, wherein the actuator is displaced from the valve seat, when F_{lift} is greater than F_{bias}.

47. The valve of claim 45 or 46, wherein the actuator starts returning to the valve seat, when F_{lift} is smaller than F_{bias}.

48. The valve of claim 41, wherein the spring constant is at least in part determined by one or more of: spring wire diameter, diameter of spring coil, number of spring coils, spring pitch, and material.

49. The valve of claim 48, wherein the spring constant is smaller than 0.05 N/mm.

50. The valve of claim 48, wherein the spring constant is between 0.005 to 0.02 N/mm.

51. The valve of claim 30, wherein the outlet member is used to adjust the compression of the biasing member, by adjusting a distance of the outlet member with respect to the actuator.

52. The valve of claim 51, wherein the compression of the biasing member is adjusted by twisting the outlet member in a first or a second direction, wherein when twisting the outlet member in the first direction, the outlet member moves closer to the actuator and compresses the biasing member further, and when twisting the outlet member in the second direction, the outlet member moves further away from the actuator and reduces the compression of the biasing member.

53. The valve of claim 52, wherein the twisting of the outlet member adjusts the predetermined range of pressures which the valve is configured to regulate.

54. The valve of any one of preceding claims, wherein the pressure of the gas within the respiratory system is at least in part determined by flow variations caused by unintentional leaks and/or patient's breathing, which causes the pressure to be lower or higher than a targeted PEEP pressure to be delivered to the patient.

55. The valve of any one of preceding claims, wherein the valve is arranged to compensate for pressure variation caused by unintentional leaks, by reducing the gas flow path size through the valve.

56. The valve of any one of preceding claims, wherein the valve is arranged to compensate for pressure variation caused by patient's breathing, by allowing a variable portion of the gas within the respiratory system to flow through the valve and exit the respiratory system.

57. The valve of any one of preceding claims, wherein the respiratory system is configured to deliver a flow rate of 5-15 L/min of breathable gas to a patient when delivering respiratory therapy.

58. The valve of any one of preceding claims, wherein the predetermined range of pressure is between 5 and 15 cm H₂O when delivering PEEP.

59. The valve of any one of preceding claims, wherein the selected pressure level is within a range of 6 to 10 H₂O, or 3 to 7 cm H₂O, or 4 to 6 cm H₂O, or 4.5-5.5 cmH₂O.

60. The valve of claim 59, wherein the movement of the actuator is able to regulate the pressure of the breathable gas within the respiratory system by a variation of −2 to +2 cm H₂O, or −1 to +1 cm H₂O, or −0.5 to +0.5 1 cm H₂O.

61. The valve of any one of preceding claims, wherein the valve is configured to be removably attached to a venting orifice of the respiratory system.

62. The valve of claim 61, wherein venting orifice is provided in a T-piece device.

63. The valve of claim 61, wherein the venting orifice is provided in an expiratory conduit of a CPAP device.

64. The valve of any one of preceding claims, wherein the valve comprises an adaptor, allowing it to be detachably coupled to the respiratory system.

65. The valve of any one of preceding claims, wherein the valve is configured to be detachably coupled to a T-piece device.

66. The valve of any one of preceding claims, the valve is configured to be detachably coupled to an expiratory conduit of a CPAP device.

67. A device for facilitating regulating pressure of gases supplied to a patient, the device comprising:
a housing defining a chamber, the housing having an inlet configured for connection with a gas flow source providing a flow of gases to the chamber, an outlet configured to direct gases out from the chamber, and a vent comprising an actuator for controlling venting of gases from the chamber through the vent;
a biasing member which biases the valve member towards a seated position;
wherein the vent and the actuator are mutually adapted so that the actuator has an exposed area which is exposed to the gases in the chamber, wherein the biasing member has a spring constant selected relative to the exposed area of the actuator whereby the actuator remains in a seated position until pressure of the gases in the chamber exceeds a selected pressure level.

68. A pressure regulating device for facilitating regulating pressure of gases supplied to a patient, the device comprising:
a housing defining a chamber, the housing comprising an inlet couplable with a flow source providing a flow of gases to the chamber, an outlet couplable with a patient interface for supplying gasses to the patient from the chamber, and a vent comprising an actuator for controlling venting of gases from the chamber through the vent;
a biasing member which biases the actuator towards a seated position whereby the actuator remains in the seated position until pressure of the gases in the chamber exceeds a selected pressure level, and
an outlet member associated with the vent, the outlet member comprising a supporting member aligned with a venting axis, the actuator having an orifice formed therein which accommodates the supporting member so that the actuator moves along the supporting member when moving relative to the seated position.
